(19) United States
(12) Patent Application Publication
(10) Pub. No.: US 2022/0304929 A1
(43) Pub. Date: Sep. 29, 2022

(54) LIPIDS FOR DELIVERY OF CHARGED MATERIAL, FORMULATIONS THEREOF AND METHOD FOR MAKING SAME

(71) Applicants: INTEGRATED NANOTHERAPEUTICS INC., Burnaby, BC (CA); THE UNIVERSITY OF BRITISH COLUMBIA, Vancouver, BC (CA)

(72) Inventors: Joshua Zaifman, Burnaby, BC (CA); Sam Chen, Burnaby, BC (CA); Yuen Yi Tam, Burnaby, BC (CA); Marco A. Ciufolini, Burnaby, BC (CA)

(21) Appl. No.: 17/634,649

(22) PCT Filed: Aug. 11, 2020
(86) PCT No.: PCT/CA2020/051098

§ 371 Date: Feb. 11, 2022

Related U.S. Application Data
(60) Provisional application No. 62/885,677, filed on Aug. 12, 2019.

(51) Int. Cl. A61K 9/127; C07C 69/732; C07C 69/675; C07C 219/06; C07C 309/67; C07C 309/68

(52) U.S. Cl. CPC A61K 9/1272; C07C 69/732; C07C 69/675; C07C 219/06; C07C 309/67; C07C 309/68

(57) ABSTRACT

Disclosed herein is a lipid having a net charge at physiological pH, and being covalently attached to a lipid moiety. The lipid moiety comprises a hydrocarbon structure having two or more linked hydrocarbon chains, optionally having cis or trans C═C, at least one of said chains being covalently attached to the head group optionally via the linker region. The hydrocarbon chains are bonded to one another at a branch point at an internal carbon of the chain attached to the linker region, which branch point comprises a functional group having an electronegative atom. The hydrocarbon chains each have between 1 and 40 carbon atoms, wherein the hydrocarbon structure in total comprises between 10 and 150 carbon atoms. Advantageously, the hydrocarbon structure may assume a generally flared shape for enhanced delivery of cargo molecules. Further provided are delivery vehicles comprising the lipids.

### TECHNICAL FIELD

Provided herein are lipids, formulations of such lipids and methods for preparing same. The lipids and formulations thereof are useful for the delivery of charged material, including but not limited to nucleic acid and peptides.

### BACKGROUND

The intracellular delivery of nucleic acids or other charged molecules such as peptides is facilitated by their incorporation into a delivery system such as a lipid nanoparticle (LNP). For example, ionizable lipid is the primary lipid component responsible for the efficient encapsulation of nucleic acids during the LNP manufacturing process. Moreover, the ionizable or cationic lipid facilitates the subsequent controlled release of the nucleic acid from endosomes after uptake by endocytosis in target cells.

It has been proposed that transfection or gene-delivery activity depends on the chemical structure of the ionizable lipid, such as a cationic lipid (Semple, S. C., et al., Rational design of cationic lipids for siRNA delivery. Nat Biotechnol, 2010, 28(2): p. 172-6). In particular, the lipophilic portion should bear a non-cylindrical shape. An example is a lipid having a small ionizable headgroup and a hydrocarbon moiety that widens or flares outwardly from the head group.

Currently, there is a need for ionizable or charged lipid structures of a desired shape that can be prepared using simple or convenient methods. The ability to provide such lipids could significantly advance the clinical development of treatments reliant on the delivery of nucleic acids or other charged molecules to target cells.

The compositions and methods of the present disclosure seek to address the foregoing problem and/or to provide useful alternatives to what has previously been described in the art.

### SUMMARY

Provided herein is an ionizable or cationic lipid having a hydrocarbon structure that facilitates the delivery of charged material. The charged material may be negatively charged material, such as a nucleic acid, or positively charged material. The lipids described herein can be prepared in a modular manner to provide tailored hydrocarbon structures of a variety of non-cylindrical shapes to facilitate the delivery of various negatively or positively charged material.

Disclosed herein is a lipid comprising a head group having a net charge at physiological pH, and being covalently attached via an optional linker region to a lipid moiety. The lipid moiety comprises a hydrocarbon structure having two or more linked hydrocarbon chains, optionally having cis or trans C═C, at least one of the chains being covalently attached to the ionizable head group optionally via the linker region. The hydrocarbon chains are bonded to one another at a branch point at an internal carbon of the chain attached to the head group via an optional linker region, which branch point comprises an X1 functional group described further herein having an electronegative atom. The hydrocarbon chains each have between 1 and 40 or 1 and 30 carbon atoms, wherein the hydrocarbon structure in total comprises between 10 and 150 carbon atoms.

According to one embodiment, there is provided a charged lipid, including a cationic or anionic lipid comprising: a head group having a net charge at physiological pH, and being covalently attached via an optional linker region to a lipid moiety; the lipid moiety comprising a hydrocarbon structure having two or more linked hydrocarbon chains, optionally having cis or trans C═C, at least one of said chains being covalently attached to the ionizable head group via the linker region, and wherein the hydrocarbon chains are bonded to one another at a branch point at an internal carbon of the hydrocarbon chain attached to the linker region, which branch point comprises an X1 functional group, the X1 functional group being selected from: —OC(O)—, —C(O)O—, —O—, —NR1-, —C(O)N(R1)-, N(R1)C(O)—, —OC(O)O—, —OC(O)N(R1)-, —N(R1)C(O)O—, —S—, —S—S—, —C(R1)=N—N—C(O)—, —C(O)—N—N═C(R1), —ON═C(R1)-, or —C(R1)=NO—, wherein the hydrocarbon chains each have between 1 and 30 carbon atoms, wherein the hydrocarbon structure in total comprises between 10 and 150 carbon atoms, and wherein R1 is independently selected from hydrogen, optionally substituted alkyl, alkenyl, alkynyl, aryl, cycloalkyl, cycloalkylalkyl, or heterocycle.

In one embodiment, the two or more linked hydrocarbon chains have a structure of Formula III:

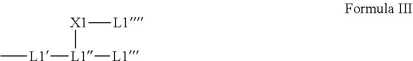

wherein L1′, L1″, L1′″ and L1″″ are independently selected from hydrocarbon chains having 1 to 30 atoms, optionally comprising one or more cis or trans C═C bond and L1′ is the hydrocarbon chain that is covalently attached to the linker region.

According to a further embodiment, the hydrocarbon structure further comprises a hydrophobic chain, L2, bonded to the head group via the linker region and wherein L2 is a hydrocarbon chain having 1 to 30 carbon atoms, optionally with a cis or trans C═C bond, or wherein L2 has the structure of Formula IIIa:

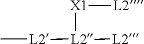

wherein L2′, L2″, L2′″ and L2″″ are independently selected from hydrocarbon chains having 1 to 30 atoms, optionally comprising one or more cis or trans C═C bond, and wherein L2′ is covalently attached to the linker region.

In yet a further embodiment, the hydrocarbon further comprises a hydrocarbon chain, L3, bonded to the head group via the linker region and wherein L3 has 1 to 30 carbon atoms, optionally with a cis or trans C═C bond, or has the structure of Formula IIIa:

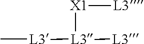

wherein L3′, L3″, L3′″ and L3″″ are independently selected from hydrocarbon chains having 1 to 30 atoms, optionally comprising one or more cis or trans C═C bond, and wherein L3′ is covalently attached to the linker region.

In a further embodiment, the lipid may further comprise 1 to 10 side chains S bonded to L1 via an X1 linkage, each side chain has 1 to 30 atoms, optionally with a cis or trans C═C bond, or has the structure of Formula IIIb:

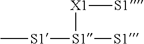

wherein S1′, S1″, S1′″ and S1″″ are independently selected from hydrocarbon chains having 1 to 30 atoms, optionally comprising one or more cis or trans C═C bond and wherein S1′ is bonded to a carbon of L1.

According to a further embodiment, the lipid may further comprise 1 to 10 side chains S bonded to L2 via an X1 linkage, each side chain has 1 to 30 atoms, optionally with a cis or trans C═C bond, or has the structure of Formula IIIb:

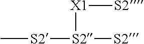

wherein S2′, S2″, S2′″ and S2″″ are independently selected from hydrocarbon chains having 1 to 30 atoms, optionally comprising one or more cis or trans C═C bond, and wherein S2′ is bonded to a carbon of L2.

In a further embodiment, the lipid further comprises 1 to 10 side chains S bonded to L3 via an X1 linkage, each side chain has 1 to 30 atoms, optionally with a cis or trans C═C bond, or has the structure of Formula IIIb:

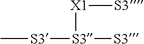

wherein S3′, S3″, S3′″ and S3″″ are independently selected from hydrocarbon chains having 1 to 30 atoms, optionally comprising one or more cis or trans C═C bond, and wherein S3′ is bonded to a carbon of L3.

In an alternative embodiment, one or more occurrences of X1 are biodegradable.

In yet a further embodiment, at least the hydrocarbon chain bonded to the head group via the linker region is derived from a lipid having one or more reactive groups selected from a hydroxyl, amino, and/or an amide bonded to an internal carbon atom thereof to serve as a scaffold carbon chain in the hydrocarbon structure and at least one other hydrocarbon chain in the hydrocarbon structure is derived from an acyl lipid, and wherein the X1 linkage is formed by reaction of the reactive group on the scaffold carbon chain with the carboxylic acid of the acyl chain.

In a further embodiment, the lipid is a di-hydroxy lipid.

The head group may impart to the lipid a pKₐ between 5.0 and 9.0 or a pKₐ of between 5.5 and 8.0. In an alternative embodiment, the head group has a structure of Formula I.

In a further embodiment, the linker region bonded to the hydrocarbon structure has a structure of Formula IIa or IIb.

In a further embodiment, the hydrocarbon structure is non-cylindrical in shape.

In a further embodiment, the lipid is capable of assembling into a lipid nanoparticle in combination with other lipids in aqueous solution.

In a further embodiment, the other vesicles forming lipids include phosphatidylcholine, phosphatidylglycerol, phosphatidylserine, phosphatidylethanolamine, phosphatidic acid, ceramides, sphingomyelin or a hydrophilic polymer-lipid conjugate.

Further provided is a drug delivery vehicle formulation comprising the lipid of any one of the foregoing embodiments incorporated in a lipid bilayer or monolayer thereof and comprising a nucleic acid or peptide.

In one embodiment, the nucleic acid is a small interfering RNA, a small activating RNA, a messenger RNA, a microRNA, an antisense oligonucleotide, a ribozyme, an aptamer, a plasmid, a circular DNA, a linear DNA, an antagomir, an anti-miRNA oligonucleotide or an miRNA mimic.

In a further embodiment, the drug delivery vehicle formulation comprises a lipid nanoparticle (LNP).

Further provided is a convenient method for preparing such lipids.

Such embodiment encompasses a method of preparing a hydrocarbon structure of a charged lipid for use in delivering a molecule of interest, the method comprising:

(i) providing a hydrocarbon chain having a reactive group or groups on an internal carbon thereof, the reactive group comprising an atom selected from O, P, N and/or S; and
(ii) conjugating the hydrocarbon chain to one or more acyl chains via the reactive group or groups to produce the hydrocarbon structure, and wherein the hydrocarbon structure is non-cylindrical in shape.

In one embodiment, the reactive group or groups form a respective X1 linkage upon conjugation a respective one of the acyl chains.

Further provided is a lipid produced by the foregoing method.

Further provided is a charged lipid comprising a branched lipid moiety L having the structure of Formula I:

A-(V)ₘ-Z-L, wherein Formula I:
A is a head group that is charged at physiological pH;
(V)m is an optional —(CR1R2)-, and m is 1 to 10 or 2 to 6, wherein R1 and R2 are each independently: hydrogen, optionally substituted alkyl, alkenyl, alkynyl, aryl, cycloalkyl, cycloalkylalkyl, or heterocycle or independently selected optionally substituted mono-, bi-, or tri-cyclic carbon ring or heteroatom ring having 4 to 12 ring atoms; and
Z-L has a structure of Formula II, IIa or IIb below:
   Formula II linear linker structure:

X1-L_{b},

wherein X1 is optional and X1 is selected from an ether, ester and carbamate group; and
L_{b} is a branched lipid of Formula IIIc;
Formula IIa branched linker structure:

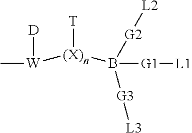

W is optional;
W, if present, is an X1 linkage, N—C(O), N—C(O)O, or N—OC(O);
wherein W is optionally substituted with D, which is an optionally substituted alkyl, alkenyl, alkynyl, aryl, cycloalkyl, cycloalkylalkyl, or heterocycle;
each occurrence of (X)ₙ is an independently selected —(CR1R2)-; n of (X)ₙ is 0 to 10; and T is optional and is an alkyl, alkenyl, alkynyl, aryl, cycloalkyl, cycloalkylalkyl, or heterocycle and wherein T is optionally substituted;
B is a carbon atom linked to L1 and L2 via respective G1 and G2;
wherein G1 and G2 are independently selected from an X1 and wherein each of G1 and G2 is independently optionally preceded and covalently bonded to a (G)ᵤ, wherein G is an independently selected —(CR1R2)- wherein R1 and R2 are each independently: hydrogen, optionally substituted alkyl, alkenyl, alkynyl, aryl, cycloalkyl, cycloalkylalkyl, or heterocycle and u is 0 to 16;

wherein G3 is optional and is selected from X1 and optionally preceded and covalently bonded to the (G)ᵤ;

L1 is a branched hydrocarbon of Formula IIIc;

L2 is a hydrocarbon chain having 1 to 20 carbon atoms and 0 to 2 cis or trans double bonds or has the structure of Formula IIIc;

L3 if present is hydrogen, a linear or branched hydrocarbon chain having 1 to 20 carbon atoms and 0 to 2 cis or trans double bonds or has the structure of Formula IIIc;

Formula IIb ring structure:

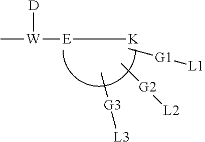

wherein the curved line represents a ring and E and K depict atoms that partially form the structure of the ring, which ring is a substituted or unsubstituted ring having 3 to 8 ring atoms;
wherein at least one of L1, L2 and L3 are bonded to a single atom in the ring, optionally via a respective G1, G2 and G3, wherein each of G1, G2 and G3 is independently optionally preceded and covalently bonded to a (G)ᵤ;
wherein L1 and optionally L2 and/or L3 of Formula IIb have the structure of Formula IIIc:

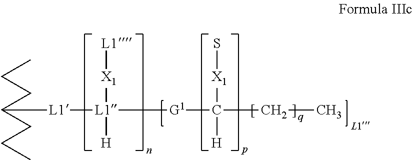

wherein an L backbone is denoted by L1′-L1″-G1-CH—[CH₂]_{q}—CH₃, and wherein the total number of carbon atoms in the L backbone is 10 to 30;

L1′ is a linear hydrocarbon chain and has 5-20, 6-20, 7-20, 8-20, 5-12, 5-10, 5-9, 6-12, 6-10, 6-9, 7-12, 7-10, or 7-9 carbon atoms and 0-3 cis or trans double bonds;

L1″ is a carbon atom;

L1″′ is depicted by G1-CH—CH₂—CH₃ and wherein G¹ is a hydrocarbon chain of 0-4 carbon atoms, optionally having one cis or trans double bond;

wherein n is 0 to 4;
wherein p is 1 to 4;
wherein n+p is 1 to 4;
q is 0 to 20;
each X1 is independently selected from an ether, ester and carbamate group;
wherein each S and L1″″ hydrocarbon side chain is independently:
(a) a linear or branched terminating hydrocarbon chain with 0 to 5 cis or trans C═C and 1 to 30 carbon atoms and conjugated to one of a respective X1 at any carbon atom in its hydrocarbon chain thereof; or
(b) a branched hydrocarbon structure of Formula IIIc,
   wherein the total number of L1″″ and S hydrocarbon chains in Formula IIIc is 1 to 10, 1 to 9, 1 to 8, 1 to 7, 1 to 6, 1 to 5, 1 to 4 or 1 to 3;
   wherein each one of the L1″″ and S hydrocarbon chains in the lipid moiety is optionally substituted with a heteroatom, with the proviso that no more than 2 heteroatoms are substituted in the hydrocarbon chains.

In another embodiment, Z-L of Formula I has the structure of Formula II (linear linker structure):

X1-L_{b};

wherein L1′ of Formula IIIc has 5 to 9 carbon atoms and has 0 to 2 cis or trans double bonds;
wherein G¹ of Formula IIIc is absent, CH₂ or CH₂CH═CH, and wherein the double bond is cis or trans;
wherein L1″″ and S of Formula IIIc are independently selected from a hydrocarbon with 0-5 cis or trans CH═CH and 2 to 18 carbon atoms;
wherein a scaffold backbone of Formula IIIc is represented by CH₂-L1″-G1-CH—CH₂—CH₃ (L1′″ is 8 to 30 carbon atoms; and
wherein q is 1 to 9.

In another embodiment (V)ₘ of Formula I is (CH₂)ₘ, wherein m is 1 to 20;

Z-L has the structure of Formula IIa (branched linker structure):

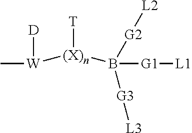

wherein W is an ether, ester or carbamate group (in any orientation) and D is absent, and (X)ₙ is (CH₂)ₙ, wherein n is 1 to 10;
wherein G1 and G2 are present and are preceded and covalently bonded to a respective (G)ᵤ, wherein (G)ᵤ is CH₂;
wherein G3-L3 is present and is a hydrocarbon selected from CH₃ and CH₂CH₃; or wherein G3-L3 is CH₂X1L3 and L3 is a linear or branched hydrocarbon chain having 1 to 20 carbon atoms and 0 to 2 cis or trans double bonds or has the structure of Formula IIIc.

In yet a further embodiment, Z-L of Formula I has the structure of Formula IIb:

Formula IIb ring structure:

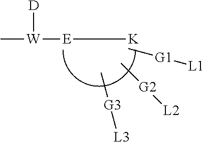

wherein the curved line represents a ring and E and K depict atoms that partially form the structure of the ring, which ring is a substituted or unsubstituted carbon ring having 3 to 6 ring atoms.

In one embodiment, the ring comprises 3 or 5 carbon atoms. In a further embodiment, at least L1 and L2 are present and are attached to the ring via respective G1 and G2 groups and wherein each G1 and G2 group is optionally preceded by a Gu, wherein u is 0 to 10 or 0 to 6.

The head group A in Formula I may be selected from:

(i) ionizable cationic moieties selected from the group consisting of:

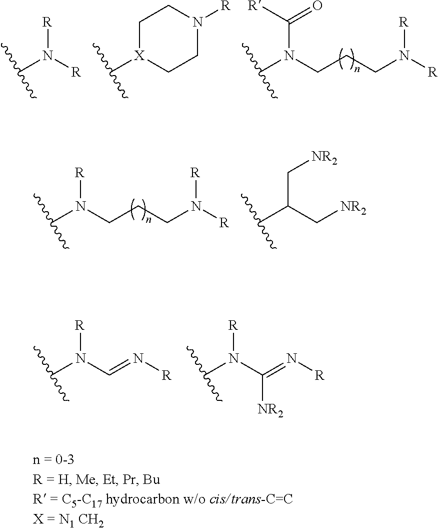

(ii) permanently charged moieties selected from the group consisting of:

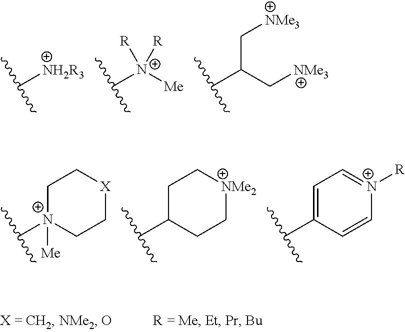

(iii) ionizable anionic moieties selected from the group consisting of:

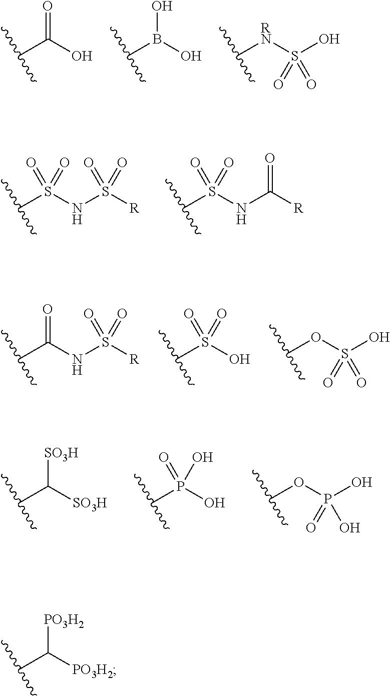

or
(iv) zwitterionic moieties selected from the group consisting of:

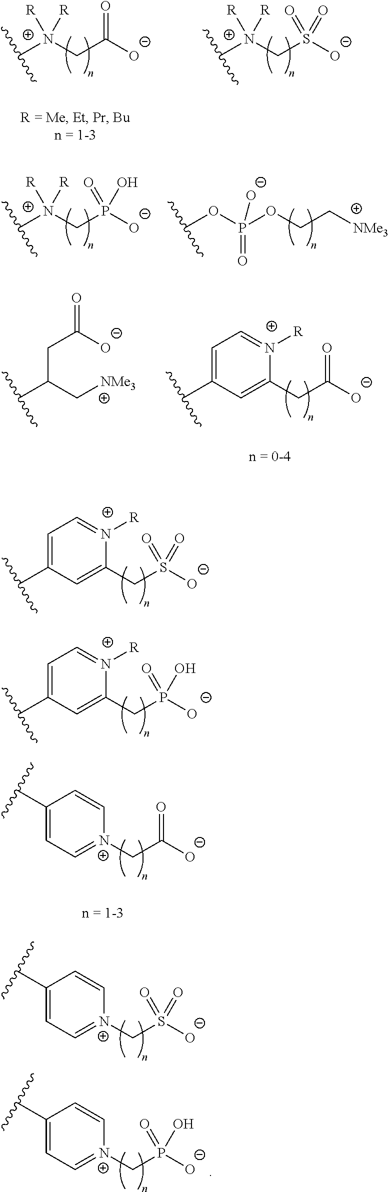

In one embodiment, the hydrocarbon structure L of Formula I is non-cylindrical in shape.

In a further embodiment the lipid is capable of assembling into a lipid nanoparticle in combination with other lipids in aqueous solution. In one embodiment, the other vesicles forming lipids include phosphatidylcholine, phosphatidylglycerol, phosphatidylserine, phosphatidylethanolamine, phosphatidic acid, ceramides, sphingomyelin or a hydrophilic polymer-lipid conjugate.

Further provided is a drug delivery vehicle formulation comprising the lipid described in any one of the foregoing embodiments that is incorporated in a lipid bilayer or monolayer thereof and comprising a nucleic acid, a protein or a peptide.

In one embodiment, the nucleic acid is a small interfering RNA, a small activating RNA, a messenger RNA, a microRNA, an antisense oligonucleotide, a ribozyme, an aptamer, a plasmid, a circular DNA, a linear DNA, an antagomir, an anti-miRNA oligonucleotide or an miRNA mimic.

In one embodiment, the drug delivery vehicle comprises a charged peptide.

In a further embodiment, the drug delivery vehicle is a lipid nanoparticle (LNP).

Various non-limiting embodiments are described herein.

### BRIEF DESCRIPTION OF FIGURES

FIG. 1A depicts amino lipids encompassed by the disclosure that have a simple head group (H) that is conjugated to one hydrocarbon chain within the lipid moiety L, optionally via a linker group. The lipid chain conjugated to the head group is derived from a hydroxy-lipid, which in turn is conjugated to one or more additional lipid chains derived from hydroxy-lipids. These lipid chains are further conjugated to one or more hydrocarbon chains derived from acyl lipids.

FIG. 1B depicts amino lipids encompassed by the disclosure that are prepared with a head group that is conjugated to two hydrocarbons within the lipid moiety L, optionally via a linker group. The two hydrocarbons conjugated to the head group are derived from hydroxy lipids. Each of the two lipid chains derived from the hydroxy lipids is, in turn, conjugated to another respective lipid chain derived from a hydroxy-lipid and to these lipids are further conjugated one or more hydrocarbon chains derived from acyl lipids.

FIG. 2A shows the activity of the amino lipids in vitro as evaluated in cultured luciferase expressing 22Rv1 cells. Cells were treated with lipid nanoparticle (LNP) formulations containing ionizable lipids of the disclosure and Luciferase siRNA at concentrations of 1.0 (left bar), 0.3 (middle bar) and 0.1 μg/mL (right bar). The level of luminescence was measured post treatment. The ionizable lipids incorporated in the LNPs were 2-(dimethylamino)ethyl (±)-syn-9,10-dilinoleoxystearate (INT-A001), 3-(diethylamino)propyl (±)-syn-9,10-dilinoleoxystearate (INT-A002), 3-(dimethylamino)propyl (±)-syn-9,10-dilinoleoxystearate (INT-A003), 2-(diethylamino)ethyl (±)-syn-9,10-dilinoleoxystearate (INT-A004), 3-(dimethylamino)propyl (12R)-linoleoxyoleate (INT-A005), 3-(diethylamino)propyl (syn-9,10,12R)-trilinoleoxystearate (INT-A006), and 3-(diethylamino)propyl (±)-syn-9,10-bis(2-hexyldecanoyloxy)stearate (INT-A007).

FIG. 26 shows the activity of the amino lipids in vitro at 1 μg/mL siRNA concentration in cultured 22Rv1 cells. The level of luminescence was measured 16-24 hr post treatment and the relative luminescence is ranked from highest (left) to lowest (right).

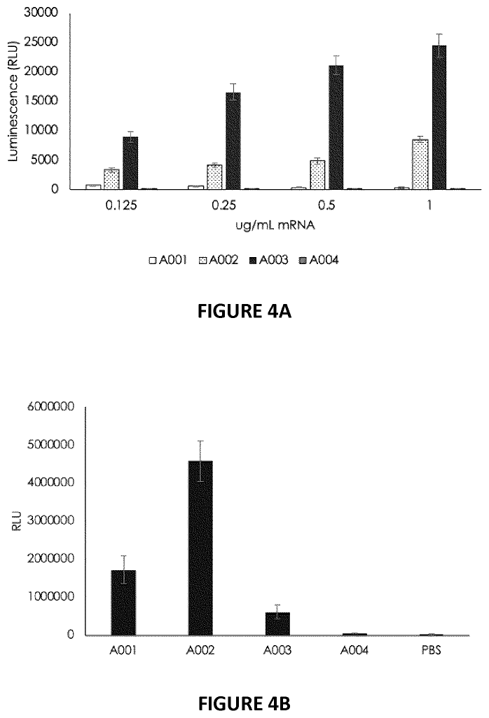

FIG. 3 shows the activity of the amino lipids in vivo as evaluated in a mouse FVII model. Lipid nanoparticle (LNP) formulations containing cationic lipids of the disclosure and FVII siRNA were injected via a tail vein in C57Bl/6 wild-type mice and the levels of FVII in plasma was measured post injection. The mice were injected with LNP-siRNA at doses of 0.03 (left bar), 0.1 (middle bar) and 0.3 mg/kg (right bar). The cationic lipids incorporated in the LNPs were INT-A001, INT-A002, INT-A003 and INT-A007.

FIG. 4A shows the activity of the ionizable lipids in vitro as evaluated in cultured HepG2 hepatocyte cells. Cells were treated with LNP formulations containing ionizable lipids of the disclosure and luciferase mRNA at concentrations of 0.125-1 μg/mL. The level of luminescence was measured post treatment. From left-to-right, the ionizable lipids incorporated in the LNPs were INT-A001 (white bar), INT-A002 (dotted bar), INT-A003 (black bar) and INT-A004 (gray bar).

FIG. 4B shows the mRNA expression in vivo in the liver of mice. Lipid nanoparticle (LNP) formulations containing cationic lipids of the disclosure and Luciferase mRNA were injected via a tail vein in C57Bl/6 wild-type mice at mRNA dose of 1 mg/kg and the luminescence in the liver was measured post injection. From left-to-right, the cationic lipids incorporated in the LNP-mRNA were INT-A001, INT-A002, INT-A003 and INT-A004. Untreated animals were injected with phosphate buffered saline (PBS).

### DETAILED DESCRIPTION

### Structure of Lipid Used for Delivery of Charged Material

The lipids described herein have a head group A and a lipid moiety L having a hydrocarbon structure described below. The head group A is charged at physiological pH and in some embodiments is ionizable, although permanently charged groups are encompassed by the disclosure as well. The head group H may contain other charged groups at physiological pH, but has a net overall positive or negative charge at physiological pH. The charged lipid may be mono-valent or multi-valent.

The lipid moiety L is generally composed of a hydrocarbon structure having a carbon chain or chains that function as a scaffold to conjugate additional hydrocarbon chains or groups within the hydrocarbon structure. In another advantageous embodiment, the lipid moiety L is at least partially comprised of hydrocarbon chains derived from hydroxy or other lipids with functional groups and serves as the scaffold component of the hydrocarbon structure. The hydrox-lipid(s) may, in turn, be conjugated to one or more hydrocarbon chains derived from acyl lipids.

As used herein, a “scaffold carbon chain” is a carbon chain that provides such a scaffold function by a covalent conjugation to another hydrocarbon chain in the lipid moiety L via a functional group as described herein (e.g., an “X1 functional group”, “X1 linkage” or other similar convention used herein). In one example, the X1 functional group includes a group having electronegative atoms, such as N, O, S or P as an atom in the group, or optionally as a sole atom in the group, and that provides a covalent linkage of the scaffold carbon chain to one or more hydrocarbon chains, including another scaffold carbon chain. An example of a suitable X1 functional group is an ester, although other groups can be readily envisioned by those of skill in the art, including ether and carbamate groups.

The scaffold carbon chain may be derived from a precursor lipid having a hydroxyl group as described herein. Such lipids are commonly referred to as hydroxy lipids and are either naturally occurring or can be synthesized in the laboratory. In order to create an ester group, for example, the hydroxyl group of the hydrocarbon chain can be reacted with a carboxylic acid on another hydrocarbon chain via a condensation reaction. However, the method of making the hydrocarbon structure is not limited to any particular preparation method since a variety of different synthesis routes are contemplated herein.

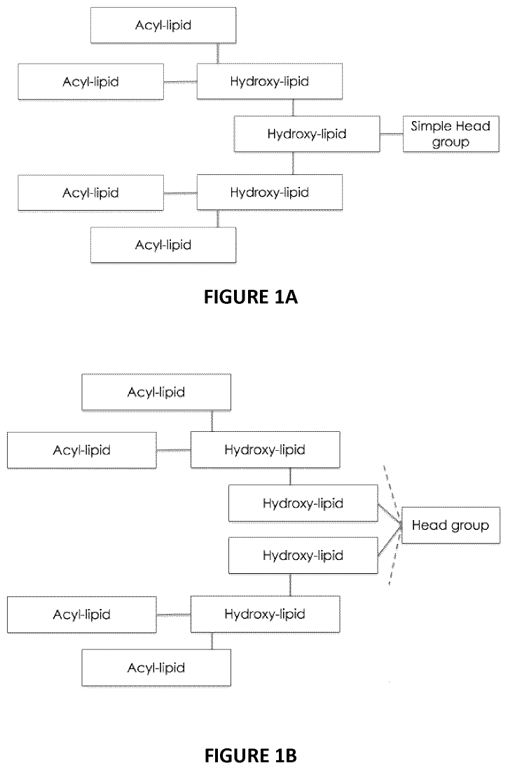

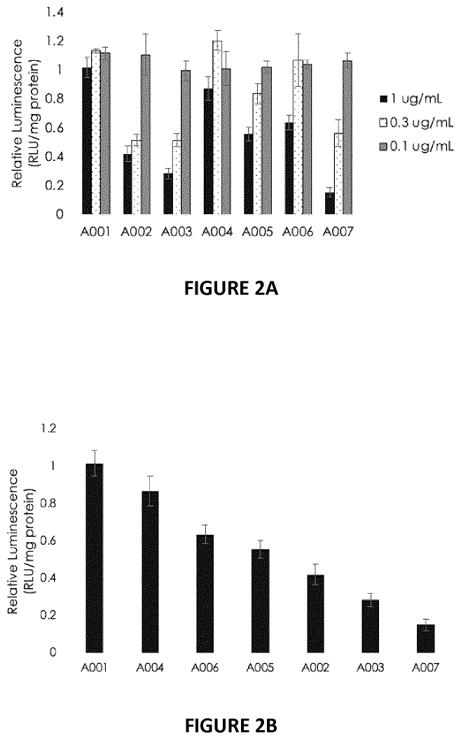

Examples of structures of charged lipids derived from hydroxylated lipids as scaffolds are described in FIGS. 1A and 1B. The lipid moiety (L) may be linked to the head group A via 1, 2 or 3 hydrocarbon groups within the lipid moiety (L).

FIG. 1A depicts charged lipids encompassed by the disclosure that have a simple head group (A) that is conjugated to one hydrocarbon within the lipid moiety L, optionally via a linker group. The first lipid chain in this embodiment is derived from a hydroxy-lipid. The hydroxy-lipid, in turn, may be conjugated to one or more additional lipid chains derived from hydroxy-lipids. These lipid chains are further conjugated to one or more hydrocarbon chains derived from acyl lipids.

FIG. 1B depicts charged lipids encompassed by the disclosure that are prepared with a head group (A) that is conjugated to two hydrocarbons within the lipid moiety L, optionally via a linker group. In this example, the two hydrocarbons conjugated to the head group are derived from hydroxy lipids. Each of the two lipids is in turn conjugated to another respective lipid derived from a hydroxy-lipid and to these lipids are further conjugated one or more hydrocarbon chains derived from acyl lipids.

It will be understood, however, that FIG. 1 is merely illustrative of select embodiments and should not be construed as limiting in any way. For example, the scaffold carbon chains alternatively could be produced from fatty acid amines, fatty acid amides and/or branched fatty acid esters.

In one example of the disclosure, the lipid comprises a head group that has a net positive or negative charge at physiological pH, and is covalently attached via a linker region (also referred to herein as a “linker”) to a lipid moiety, the lipid moiety comprising a hydrocarbon structure having two or more linked hydrocarbon chains, optionally having cis or trans C═C, at least one of the chains being covalently attached to the ionizable head group via the linker region, and wherein the hydrocarbon chains are bonded to one another at a branch point at an internal carbon of the chain attached to the linker region, which branch point comprises an X1 functional group, the X1 functional group being selected from: —OC(O)—, —C(O)O—, —O—, —NR1-, —C(O)N(R1)-, N(R1)C(O)—, —OC(O)O—, —OC(O)N(R1)-, —N(R1)C(O)O—, —S—, —S—S—, —C(R1)=N—N—C(O)—, —C(O)—N—N═C(R1), —ON═C(R1)-, or —C(R1)=NO—, wherein the hydrocarbon chains each have between 1 and 30 carbon atoms, wherein the hydrocarbon structure in total comprises between 10 and 150 carbon atoms, and wherein R1 is independently selected from hydrogen, optionally substituted alkyl, alkenyl, alkynyl, aryl, cycloalkyl, cycloalkylalkyl, or heterocycle.

In a further embodiment, the hydrocarbon structure of the charged lipid has a shape that is non-cylindrical. Many phospholipids have hydrocarbon chains (formed by two fatty acid chains) linked to a glycerol backbone that form a lipophilic component that is generally cylindrical in shape. However, in certain embodiments, the hydrocarbon structure described herein forms a “flared” structure (also referred to as “frusto-conical” in shape), meaning that the hydrocarbon structure has a diameter at any point along its length that is at least 1.2, or at least 1.5, or at least 2 times greater than that of the largest diameter of the head group. Such shapes are advantageous in that they can facilitate the transfection of nucleic acids or other charged molecules to cells.

In another embodiment, the hydrocarbon structure of the charged lipid has three or more hydrocarbon chains, each of the three or more hydrocarbon chains being linked to another chain at an internal carbon thereof via the foregoing X1 linkage. In another embodiment, the lipid moiety has a hydrocarbon structure with 2 to 20 or 3 to 18 conjugated hydrocarbon chains. The hydrocarbon structure in one embodiment can be described as a lattice or matrix of connected hydrocarbon chains forming a flared, non-cylindrical structure. The connection points are optionally each biodegradable functionalities or at least 1, 2, 3, 4 or 5 of the connection points are biodegradable, as measured in vivo after administration to a patient.

While a variety of head groups are contemplated, in one example, the head group comprises an amino group that is ionizable, such as a terminal amine group. In one embodiment, the head group does not comprise a phosphate group. In another embodiment, the amine is a primary, secondary, tertiary or quaternary amine. If a quaternary amine is utilized in the head group, then the lipid may be non-ionizable, depending on the presence or absence of other ionizable groups in the head group. In another embodiment, the head group is non-zwitterionic.

It should be understood that each of the X1 linkages connecting the hydrocarbon chains in the hydrocarbon structure of L may be the same or differ. That is, each X1 can be independently selected from the X1 linkage groups listed above.

In one embodiment, the X1 linkage is selected from OC(O)—, —C(O)O—, and —O—. In a further alternative embodiment, the X1 linkage is any covalent bond that is biodegradable. The X1 linkage may also be pH sensitive, meaning cleavage is dependent on the pH of the surrounding solution.

In a further embodiment, the hydrocarbon structure is produced from one or more hydroxy lipids and one or more acyl lipids, wherein the one or more hydroxy lipids function as a scaffold carbon chain to conjugate said one or more acyl lipids.

In a further embodiment, the lipid has an apparent pKₐ between 5.0 and 9.0 or between 5.5 and 8.5 or between 5.0 and 8.0.

In yet a further embodiment, the charged lipid (e.g., cationic or ionizable lipid) described above can be formulated in a lipid nanoparticle, including, but not limited to, a liposome.

In a further embodiment, the charged lipid has the structure of Formula I:

A-(V)ₘ—Z-L, wherein Formula I:
A is a head group that is charged at physiological pH;
(V)ₘ is an optional —(CR1R2)-, and m is 1 to 10 or 2 to 6, wherein R1 and R2 are each independently: hydrogen, optionally substituted alkyl, alkenyl, alkynyl, aryl, cycloalkyl, cycloalkylalkyl, or heterocycle or independently selected optionally substituted mono-, bi-, or tri-cyclic carbon ring or heteroatom ring having 4 to 12 ring atoms; and
Z-L has a structure of Formula II, IIa or IIb below.

Formula II is a linear linker structure:

X1-L_{b},

wherein X1 is optional and X1 is selected from an ether, ester and carbamate group; and L_{b} is a branched lipid of Formula IIIc below.

In another example of the disclosure, the charged lipid is an amino lipid and has the following Formula Ia:

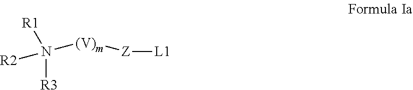

wherein R1, R2 and R3 are each independently: hydrogen, optionally substituted alkyl, alkenyl, alkynyl, aryl, cycloalkyl, cycloalkylalkyl, or heterocycle;
wherein, optionally, one of R1, R2 and R3 is absent (a lone pair), or hydrogen;
each occurrence of V is an independently selected —(CR1R2)-, and m is 1 to 10 or 2 to 6.

Z-L of Formula I or Z-L1 of the amino lipid of Formula Ia may be represented by Formula II, Formula IIa or Formula IIb depicted below:

X1-L_{b}, Formula II:

wherein X1 is optional and X1 in one embodiment is selected from an ether, ester and carbamate group; and
L_{b} is a branched lipid of Formula III, IIIa or IIIc.

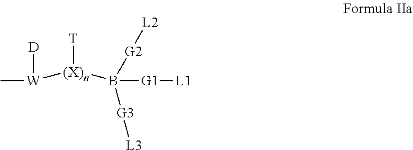

W is optional;
W, if present, is an —OC(O)—, —C(O)O—, —O—, —NR1-, —C(O)N(R1)-, N(R1)C(O)—, —NC(O)R1-, —OC(O)O—, —OC(O)N(R1)-, —N(R1)C(O)O—, —S—, —S—S—, C(R1)=N—N—C(O)—, C(O)—N—N═C(R1), —ON═C(R1), or C(R1)=NO—;
wherein W is optionally substituted with D, which is an optionally substituted alkyl, alkenyl, alkynyl, aryl, cycloalkyl, cycloalkylalkyl, or heterocycle;
each occurrence of (X)ₙ is an independently selected —(CR1R2)-, or independently selected optionally substituted mono-, bi-, or tri-cyclic carbon ring or heteroatom ring having 4 to 12 ring atoms; n of (X)ₙ is 0 to 10; and T is optional and is an optionally substituted alkyl, alkenyl, alkynyl, aryl, cycloalkyl, cycloalkylalkyl, or heterocycle;
B is a carbon, oxygen, nitrogen atom covalently attached to L1, optionally via G1; and optionally additionally attaches independently selected L2 and/or L3, optionally via respective G2 and G3 groups;
wherein G1, G2 and G3 are independently selected from —OC(O)—, —C(O)O—, —O—, —NR1-, —C(O)N(R1)-, N(R1)C(O)—, —OC(O)O—, —OC(O)N(R1)-, —N(R1)C(O)O—, —S—, —S—S—, C(R1)=N—N—C(O)—, C(O)—N—N═C(R1), —ON═C(R1), or C(R1)=NO— and wherein each of G1, G2 and G3 is independently optionally preceded and covalently bonded to a (G)ᵤ wherein G is an independently selected —(CR1R2)- and u is 0 to 10;

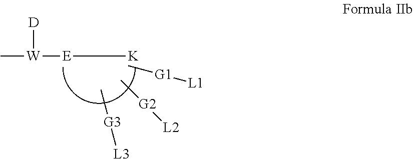

W is optional;
W, if present, is an —OC(O)—, —C(O)O—, —O—, —NR1-, —C(O)N(R1)-, N(R1)C(O)—, —NC(O)R1-, —OC(O)O—, —OC(O)N(R1)-, —N(R1)C(O)O—, —S—, —S—S—, C(R1)=N—N—C(O)—, C(O)—N—N═C(R1), —ON═C(R1), or C(R1)=NO—;
wherein W is optionally substituted with D, which is an optionally substituted alkyl, alkenyl, alkynyl, aryl, cycloalkyl, cycloalkylalkyl, or heterocycle;
wherein the curved line represents a ring and E and K depict any atoms that partially form the structure of the ring, which ring is a substituted or unsubstituted, mono-, bi-, or tri-cyclic carbon ring or mono-, bi-, or tri-cyclic heteroatom ring having 4 to 12 ring atoms;
L2 and L3 in Formula Ia and Ib are optional hydrogen atoms or hydrocarbon chains having 1 to 40 carbon atoms and optionally having one or more cis or trans C═C double bonds or have a structure of Formula IIIa (described hereinafter);
wherein two of L1, L2 and L3 are optionally bonded to a single atom in the ring represented by the curved line;
L, L_{b}, L1 and optionally L2 and/or L3 of the above formulas is a lipid moiety having the structure of Formula III described immediately below. Alternatively, the lipid moiety comprises a structure including Formula IIIa or Formula IIIc described hereinafter.

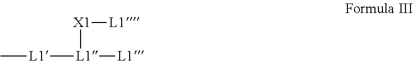

wherein L1′, L1″, L1′″ and L1″″ are independently selected from hydrocarbon chains having 1 to 30 atoms, optionally comprising one or more cis or trans C═C;
wherein X1 is —OC(O)—, —C(O)O—, —O—, —NR1-, —C(O)N(R1)-, N(R1)C(O)—, —OC(O)O—, —OC(O)N(R1)-, —N(R1)C(O)O—, —S—, —S—S—, C(R1)=N—N—C(O)—, C(O)—N—N═C(R1), —ON═C(R1), or C(R1)=NO—; and
wherein, Formula III optionally comprises 1 to 10 side chains S, each independently selected from hydrocarbon chains having 1 to 30 atoms, optionally comprising one or more cis or trans C═C or a sterol and wherein at least one S is attached covalently to a carbon atom of L1′, L1″, L1′″ and/or L″″ via a linkage that is X1.

The charged lipid (e.g., ionizable lipid) may be a mixture of enantiomers or contain a single optical isomer. As discussed above, the X1 group can be biodegradable, meaning that it can be cleaved after administration to a subject. Without being limiting, an ester bond is capable of being hydrolyzed by an esterase after administration to a patient, thereby releasing a hydrocarbon chain from the lipid. Other groups capable of being hydrolyzed by an enzyme, or in response to a pH change, are encompassed by the disclosure as well. However, it will be understood that the X1 group can be a non-biodegradable group as well.

In one embodiment, the lipid is soluble in a biocompatible alcohol, thereby facilitating its incorporation into a delivery vehicle and co-encapsulation of nucleic acid, such as a small interfering RNA, small activating RNA, messenger RNA, microRNA, antisense oligonucleotide, ribozymes, aptamer, plasmid, circular DNA, linear DNA, antagomir, anti-miRNA oligonucleotide, miRNA mimic or gene editing material.

### Head Group (A)

Lipids are often conveniently represented as having a head group (denoted herein as “A”) covalently attached to one or more hydrocarbon chains. Optionally, the head group A is attached to the hydrocarbon chain or chains by a linker region. Suitable head groups and linker regions are described below.

In one embodiment, the head group is selected from moieties that are ionizable, permanently charged or zwitterionic. The head group may impart either a positive or a negative charge to the lipid at a given pH value or range, including pH 7.4 (physiological pH). Examples of chemical groups that fall within each of these categories are as follows:

(i) ionizable cationic moieties:

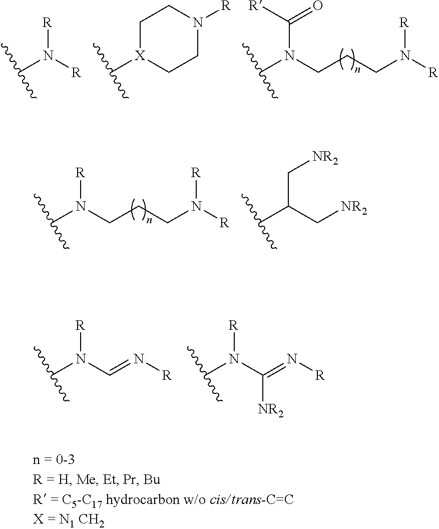

(ii) permanently charged moieties:

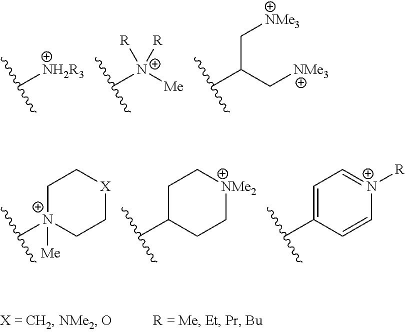

(iii) ionizable anionic moieties:

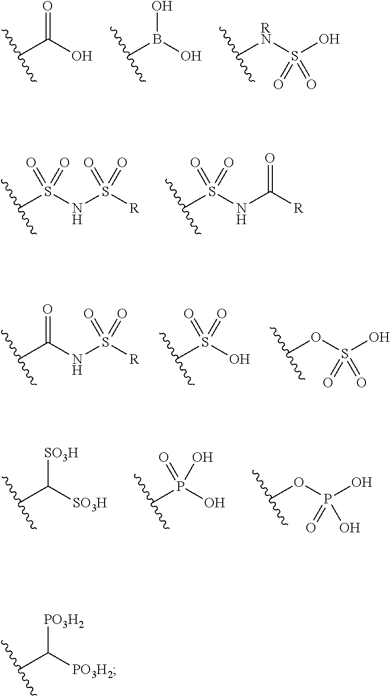

or
(iv) zwitterionic moieties:

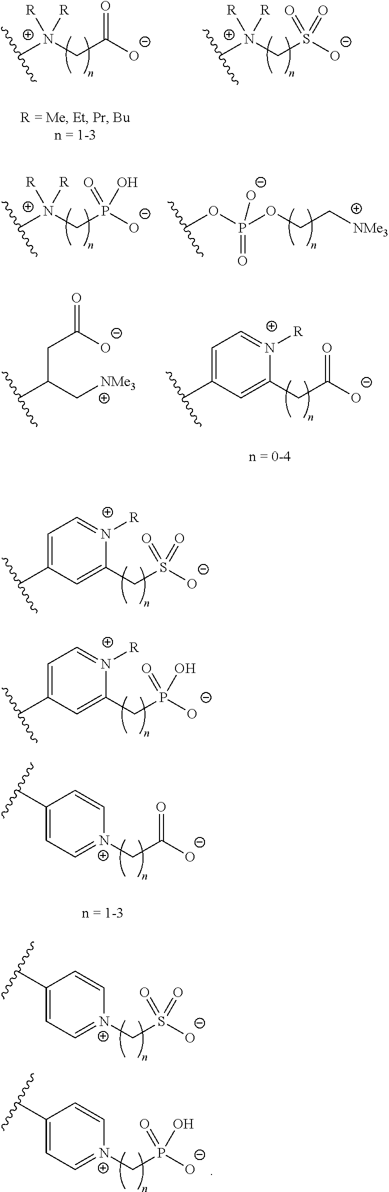

Typically, the head group A of the lipid is relatively small and charged at physiological pH. In one non-limiting embodiment, such head group comprises a terminal ionizable amine group, although although groups such as phosphate and sulfate groups are contemplated herein as well. In another embodiment, the head group has a terminal ionizable amine group that imparts an apparent pKₐ of between 5 and 8 to the ionizable lipid or a pKₐ of 5.5 to 7.5 to the ionizable lipid. It will be appreciated that the pKₐ of the amino group may be influenced by neighbouring atoms in the head group. Head groups containing sulfate or phosphate may impart an overall negative charge to the lipid.

The amine group may be a primary, secondary or tertiary amine group. The head group may contain additional amine groups, such as two or three amine groups that are the same or different.

In one embodiment, the amine portion of the head group has the following formula:

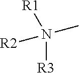

wherein R1, R2 and R3 are each independently: hydrogen, optionally substituted alkyl, alkenyl, alkynyl, aryl, cycloalkyl, cycloalkylalkyl, or heterocycle; wherein one of R1, R2 and R3 is absent (a lone pair), or hydrogen.

In those embodiments where the head group is positively charged, such group does not possess a phosphate group since this group imparts a negative charge and thus may result in a lipid that is charge neutral. However, such a group may be included in the head group provided the overall charge of the lipid is positive at physiological pH.

Optionally, the head group is conjugated to a hydrophilic polymer, including a polymer that improves circulation longevity after administration of the lipid or a formulation thereof to a patient. A non-limiting example of such a hydrophilic polymer is polyethylene glycol (PEG).

As would be appreciated by those of skill in the art, when the head group is charged, the lipid may be present as a salt. Any pharmaceutically acceptable salt is included within the scope of the disclosure.

### Optional Linker Region

In addition to the ionizable amine group, the lipid may comprise a region having one or more electronegative atoms that allow covalent attachment to 1, 2 or 3 lipid chains, at least one of which is a scaffold carbon chain. Such region in the lipid can be referred to as a linker region, or simply a “linker” and is optional since the hydrocarbon chain or chains may be linked directly to the head group.

The chemical structure of the linker may depend on the number of lipid chains that are attached to the head group, but may include within its structure an ester, ether, glyceride linker, a derivative of a glyceride linker, or a cyclic linker, including multi-membered rings composed of carbon or heteroatoms.

The linker region attached to the head group may have the following formula:

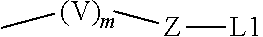

Wherein (V)ₘ—Z depicts the linker region attached at one end to the head group A and at the other end to L1, which is a hydrocarbon region.

The portion Z-L1 of the formula together may be represented by Formula II, Formula IIa or Formula IIb set forth above.

Optionally, the linker region is conjugated to a hydrophilic polymer, including a polymer that improves circulation longevity, such as polyethylene glycol (PEG).

As would be appreciated by those of skill in the art, a variety of different combinations of head-linker groups are encompassed by the present disclosure. The linker region may be characterized by the number of hydrocarbon chains attached thereto. Alternatively, the linkers may be described as linear, branched or cyclic. Examples of each category are provided below.

Non-limiting examples of head groups with linker regions that attach a single hydrocarbon chain are provided below:

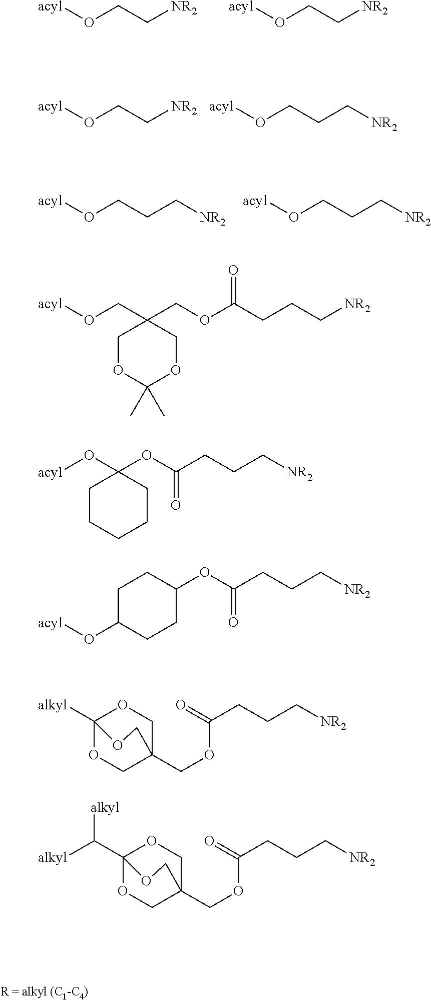

Non-limiting examples of head groups with linker regions that attach two hydrocarbon chains are provided below:

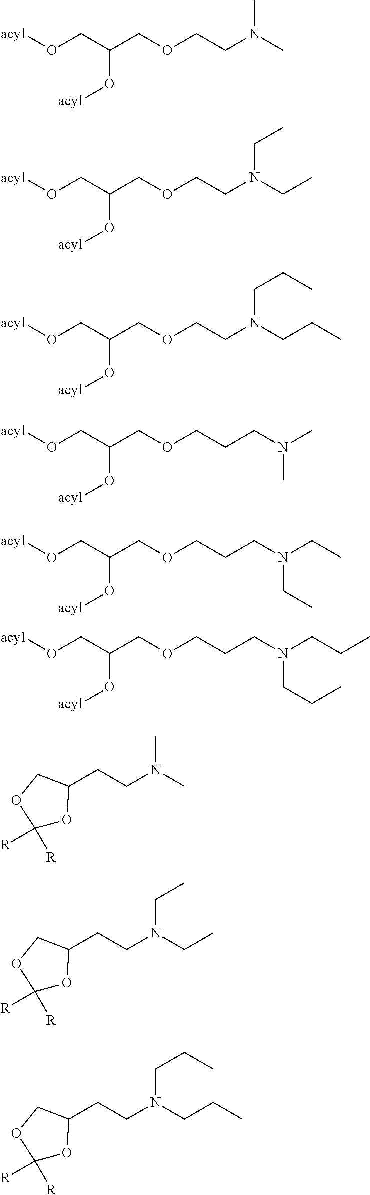

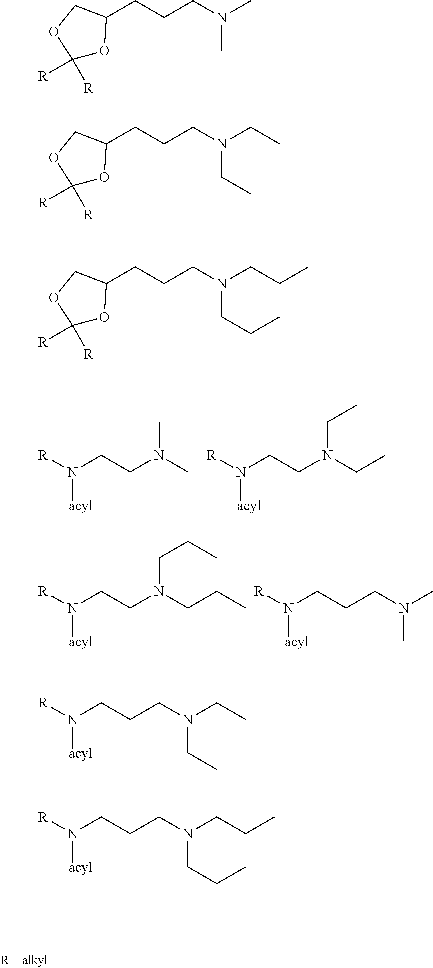

Non-limiting examples of head groups with linker regions that attach three hydrocarbon chains are provided below:

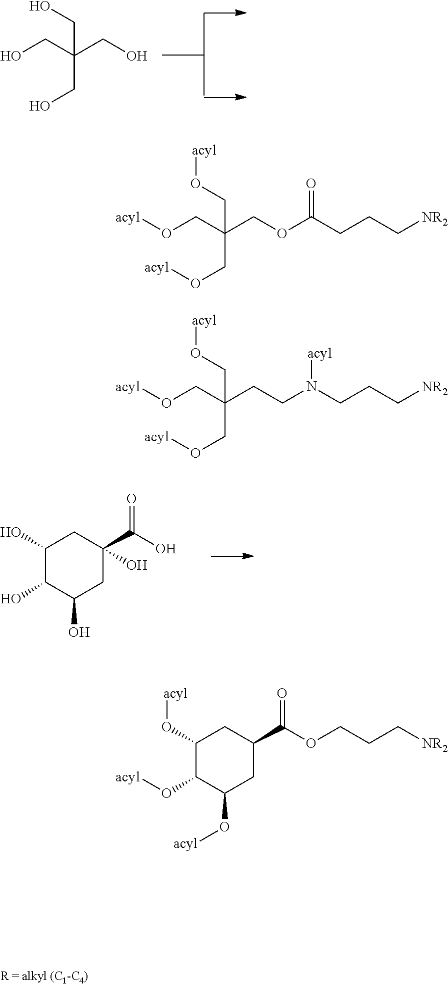

Linker regions may also be described as linear, branched or cyclic. Examples of linear, branched and cyclic linker regions are described in Formula II, IIa and IIb, respectively, set forth above.

### Lipid Moiety L

The lipid moiety L may include 1, 2 or 3 hydrocarbon chains within the hydrocarbon structure attached to the head group directly or via the linker region. In those embodiments in which only one hydrocarbon chain is attached to the head group or linker region, additional hydrocarbon side chains, S, may be linked to an internal carbon of L1.

Thus, at a minimum, in one embodiment, the head group has attached thereto, or via a linker, an L1 that is a lipid moiety having the structure of Formula III:

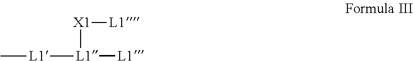

wherein L1′, L1″, L1′″ and L1″″ are independently selected from hydrocarbon chains having 1 to 30 atoms, or 1 to 20 atoms, optionally comprising one or more cis or trans C═C; and
wherein X1 is —OC(O)—, —C(O)O—, —O—, —NR1-, —C(O)N(R1)-, —N(R1)C(O)—, —OC(O)O—, —OC(O)N(R1)-, —N(R1)C(O)O—, —S—, —S—S—, C(R1)=N—N—C(O)—, —C(O)—N—N═C(R1), —ON═C(R1), or —C(R1)=NO—.

Optionally, the head group has attached thereto, optionally via the linker, an L2 that is a lipid moiety, or a further L3 lipid moiety. The optional L2 and L3 lipid moieties can be each independently selected from hydrocarbon chains having 1 to 30 atoms, or 1 to 20 atoms, optionally comprising one or more cis or trans C═C, or a sterol. L2 and/or L3 optionally may be a lipid of Formula IIIa below.

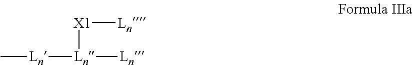

wherein Ln′ is covalently attached to the linker.

According to the nomenclature of Formula IIIa, n is 2 if L is L2 and n is 3 if L is L3. For example, for an L2 hydrocarbon, Formula IIIa adopts the following nomenclature:

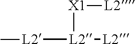

Formula III or IIIa optionally comprises 1 to 20 side chains S, depicted as (S)ₙ, wherein n is 0 to 20, each independently selected from hydrocarbon chains having 1 to 30 atoms, or 1 to 20 atoms, optionally comprising one or more cis or trans C═C, or a sterol and wherein (S)ₙ is/are attached covalently to a carbon atom of L1, L2 and/or L3 via a linkage that is X1 or covalently bonded to another side chain S in the hydrocarbon structure via such linkage. In one embodiment, the X1 linkage is biodegradable, such as an ester linkage. However, other linkages besides those that are biodegradable can be used in the practice of the invention.

For example, L1 may have a side chain S1′ conjugated to any one of L1′, L1″′ or L1″″ via an X1 linkage. Additional side chains S1″, S1′″ or S1″″ may be attached to a carbon of L1 or any S1 side chain. In a further example, if L2 has a structure of Formula IIIa above, L2 may have side chain S2′ attached to any one of L2′, L2′″ or L2″″. Additional side chains S2″, S2′″ and S2″″ may be attached to a carbon of L2 or any S2 side chain. Likewise, if L3 is present and has Formula IIIa above, it may have side chain S3′ attached to any one of L3′, L3′″ or L3″″. Additional side chains S3″, S3′″ and S3″″ may be attached to a carbon of L3 or any S3 side chain.

Examples of lipids having structures encompassed by the present disclosure are depicted in Table 1 (below). Structures A-F shown in Table 1 include head groups with linkers attached to a single hydrocarbon with X1 branch points that provide scaffold attachment points to additional S hydrocarbon chains. Structures G-J shown in Table 1 include head groups with linkers attaching two hydrocarbons, L1 and L2. The lipids depicted as Structures K-M have linkers with attachment points to three hydrocarbons, L1, L2 and L3.

The annotated Structure A below exemplifies the convention used in Formula III herein for depicting the X1 linkages and hydrophobic regions of L1. It will be understood that each occurrence of X1 is independently selected from another X1 in the structure. As discussed, the X1 groups include any suitable functional group with electronegative atoms.

The example shown is an amino lipid having a single L1 lipid hydrocarbon chain (L1′, L1″ and L1′″) according to Formula III above with a side chain S linked to a carbon of L1:

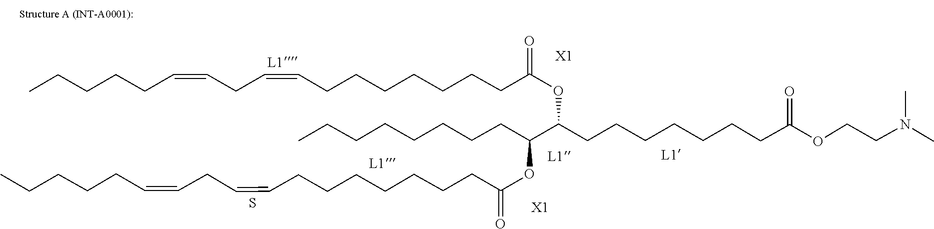

As can be seen in the Structure A (INT-A001) depicted above, L1′, L1″ and L1′″ together form a linear hydrocarbon backbone (referred to herein as a “scaffold carbon chain”) that is derived from a di-hydroxy lipid. This hydrocarbon chain, L1′-L1″-L1″′ links L1″″ to the L1″ carbon of L1′-L1″-L1″′ via an X1 linkage and a second S “side chain” hydrocarbon chain is conjugated to L1′″ via a second X1 linkage. In this structure, both X1 linkages are ester groups. However, as noted above, the X1 functional groups can be independently selected from a variety of functional groups containing O, N, P and/or S atoms.

Examples of lipids having a linker region within the head group that is attached to one, two or three hydrocarbon chains are provided in Table 1 below. For each lipid depicted, the substituents L1′/L″/L′″/L″″ of L1 of Formula III are provided, as well as the optional L2 and L3 hydrocarbon chains. Also provided is the structure of the head group and linker region as defined by Formula IIa or IIb. In each case, the X1 group is an ester that links the hydrocarbon chains to one another to form an interconnected hydrocarbon lattice structure. As can be seen from the structures below, the interconnected hydrocarbon structures of L form a generally flared or frusto-conical shape.

TABLE 1
Select amino lipids having linker region attached to one, two or three hydrocarbon
chains L1, L2 and L3 forming a flared hydrocarbon structure

| | | | |
|---|---|---|---|
| Lipid structure | 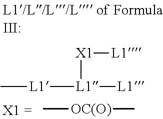 | Side chain S (if present) linked to another hydrocarbon via an ester (X1 = —OC(O)—) | Linker and head group (terminal amine) |

ONE HYDROCARBON CHAIN (L1) LINKED TO HEAD GROUP

| | | | |
|---|---|---|---|
| 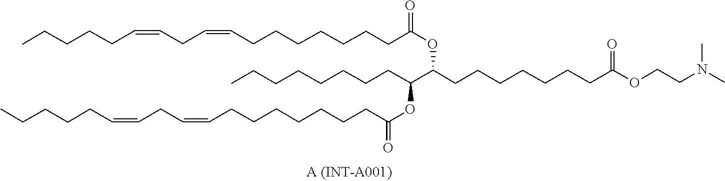 [SMILES: CCCCC/C=C\C/C=C\CCCCCCCC(=O)O[C@@H](CCCCCCCC)[C@@H](CCCCCCCC(=O)OCCN(C)C)OC(=O)CCCCCCC/C=C\C/C=C\CCCCC] | L1′: 7 carbon alkyl L1″: 1 carbon L1′′′: 9 carbon alkyl L1′′′′: 17 carbon chain with two cis C═C bonds | S: 17 carbon chain with two cis C═C bonds linked to L1′′′ | Linker: ester- containing Amine: NMe₂ |
| 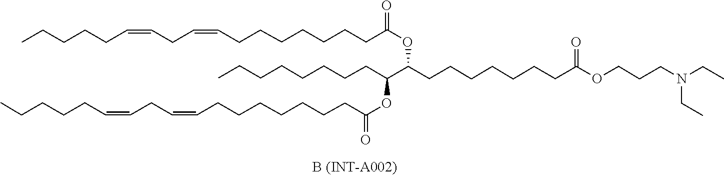 [SMILES: CCCCC/C=C\C/C=C\CCCCCCCC(=O)O[C@@H](CCCCCCCC)[C@@H](CCCCCCCC(=O)OCCCN(CC)CC)OC(=O)CCCCCCC/C=C\C/C=C\CCCCC] | L1′: 7 carbon alkyl L1″: 1 carbon L1′′′: 9 carbon alkyl L1′′′′: 17 carbon chain with two cis C═C bonds | S: 17 carbon chain with two cis C═C bonds linked to L1′′′ | Linker: ester- containing Amine: NEt₂ |
| 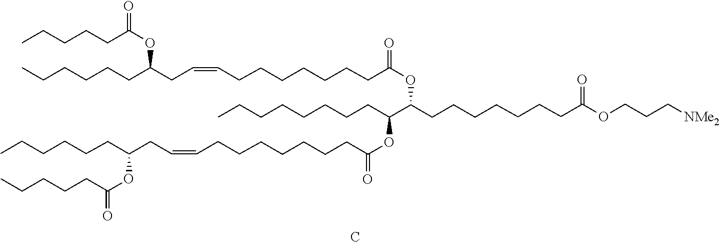 | L1′: 7 carbon alkyl L1″: 1 carbon L1′′′: 9 carbon alkyl L1′′′′: 17 carbon chain with a cis C═C bond | S1′: 17 carbon chain with one cis C═C linked to L1′′′ S1″: 5 carbon alkyl linked to S1′ S1′′′: 5 carbon alkyl linked to L1′′′′ | Linker: ester- containing Amine: NMe₂ |
| 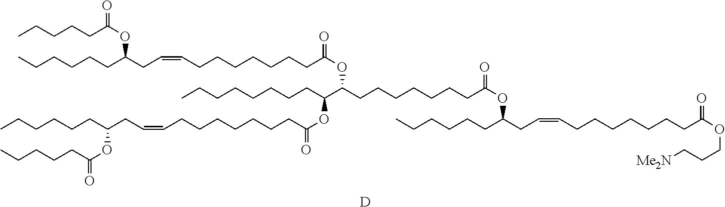 | L1′: 10 carbon chain with one cis C═C bond L1″: 1 carbon L1′′′: 9 carbon alkyl L′′′′: 19 carbon alkyl | S1′: 17 carbon chain with one cis C═C linked to L1′′′′ S1″: 5 carbon alkyl linked to S1′ S1′′′: 17 carbon chain with one cis C═C linked to L1′′′′ S1′′′′: 5 carbon | Linker: ester containing Amine: NMe₂ |
| | | alkyl linked to | |
| | | S1′′′ | |
| 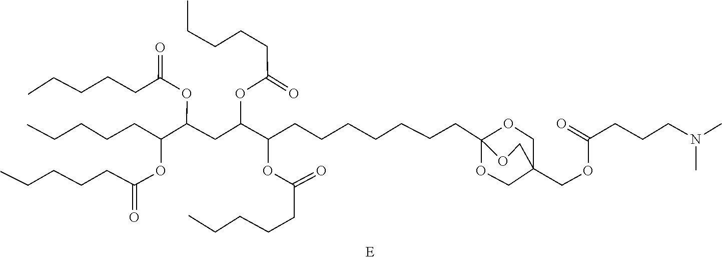 [SMILES: CCCCCC(=O)OC(CCCCC)C(CC(OC(=O)CCCCC)C(CCCCCCCC12OCC(COC(=O)CCCN(C)C)(CO1)CO2)OC(=O)CCCCC)OC(=O)CCCCC] | L1′: 8 carbon alkyl chain L1″: 1 carbon L1′′′: 9 carbon alkyl L1′′′′: 5 carbon alkyl | S1′: 5 carbon alkyl linked to L1′ S1′: 5 carbon alkyl linked to L1′′′ S1″: 5 carbon alkyl linked to L1′′′ | Linker: ester containing with tricyclic heteroatom Amine: NMe₂ |
| 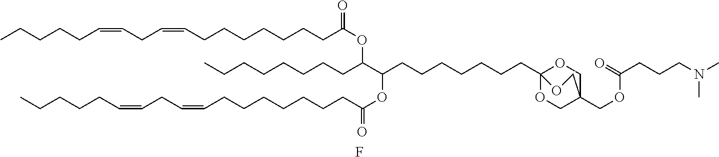 | L1′: 7 carbon alkyl L1″: 1 carbon L1′′′: 9 carbon alkyl L1′′′′: 17 carbon chain with two cis C═C bonds linked to L1′′′ via an ester | S1′: 17 carbon chain with two cis C═C bonds linked to L1′ via an X1 ester | Linker: ester containing with tri- cyclic heteroatom Amine: NMe₂ |

TWO HYDROCARBON CHAINS (L1 AND L2) LINKED TO HEAD GROUP

| | | | |
|---|---|---|---|
| 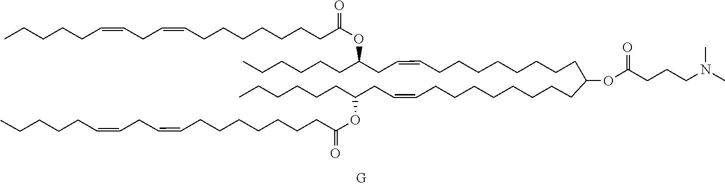 | L1′: 11 carbon chain with one cis C═C bond L1″: 1 carbon L1′′′: 6 carbon alkyl L1′′′′: 17 carbon chain with two cis C═C bonds L2′: 11 carbon chain with one cis C═C bond L2″: 1 carbon L2′′′: 6 carbon alkyl L2′′′′: 17 carbon chain with | N/A | Linker: ester- containing Amine: NMe₂ |
| | two cis C═C bonds | | |
| 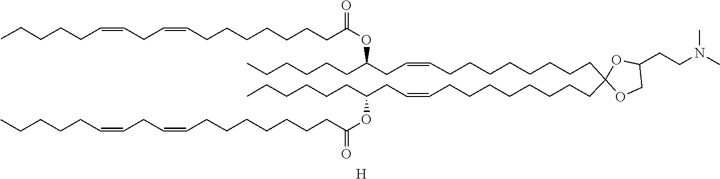 | L1′: 11 carbon chain with one cis C═C bond L1″: 1 carbon L1′′′: 6 carbon alkyl L1′′′′: 17 carbon chain with two cis C═C bonds L2′: 11 carbon chain with one cis C═C bond L2″: 1 carbon L2′′′: 6 carbon alkyl L2′′′′: 17 carbon chain with | N/A | Linker: Mono- cyclic heteroatom ring Amine: NMe₂ |
| | two cis C═C bonds | | |
| 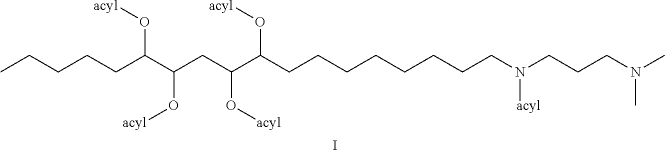 | L1′: 8 carbon alkyl chain L1″: 1 carbon L1′′′: 9 carbon alkyl chain L1′′′′: hydrocarbon of acyl chain linked via ester L2: carbon chain linked to N of linker via ester | S1′: hydrocarbon of acyl chain linked to L1′′′ S1″: hydrocarbon of acyl chain linked to L1′′′ S1′′′: | Linker: Formula IIa: W—D, (X)ₙ and L3 are absent; B═N G2 = —OC(O)— |
| | | hydrocarbon of | Amine: |
| | | acyl chain linked | NMe₂ |
| | | to L1′′′ | |
| 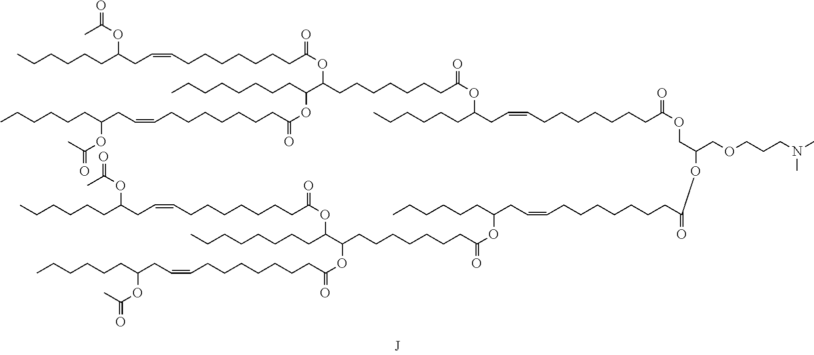 [SMILES: CCCCCCCCC(OC(=O)CCCCCCC/C=C\CC(CCCCCC)OC(C)=O)C(CCCCCCCC(=O)OC(C/C=C\CCCCCCCC(=O)OCC(COCCCN(C)C)OC(=O)CCCCCCC/C=C\CC(CCCCCC)OC(=O)CCCCCCCC(OC(=O)CCCCCCC/C=C\CC(CCCCCC)OC(C)=O)C(CCCCCCCC)OC(=O)CCCCCCC/C=C\CC(CCCCCC)OC(C)=O)CCCCCC)OC(=O)CCCCCCC/C=C\CC(CCCCCC)OC(C)=O] | L1′: 10 carbon chain with cis C═C bond L1″: 1 carbon L1′′′: 6 carbon alkyl L1′′′′: 17 carbon alkyl chain L2′: 10 carbon chain with cis C═C bond L2″: 1 carbon L2′′′: 6 carbon alkyl L2′′′′: 17 carbon alkyl chain | S1′: 17 carbon chain with cis C═C bond linked to L′′′′ S1′: 1 carbon chain linked to a carbon of S1′ S1″: 17 carbon chain with cis C═C bond linked to a carbon of S1′ S1′′′: 1 carbon chain linked to a carbon of S1″ S2′: 17 carbon chain with cis C═C bond S2″: 1 carbon chain linked to a carbon of S2′ S2′′′: 17 carbon chain with cis | Linker: Glycerol containing Formula IIa: W is —O—, D is absent and (X)ₙ is CH₂ B is C G1 is —CH₂OC(O)— G2 is —OC(O)— Amine: NMe₂ |
| | | C═C bond linked | |
| | | to a carbon of | |
| | | S2′ | |
| | | S2′′′′: 1 carbon | |
| | | chain linked to a | |
| | | carbon of S2′′′ | |

THREE HYDROCARBON CHAINS (L1, L2 AND L3) LINKED TO HEAD GROUP

| | | | |
|---|---|---|---|
| 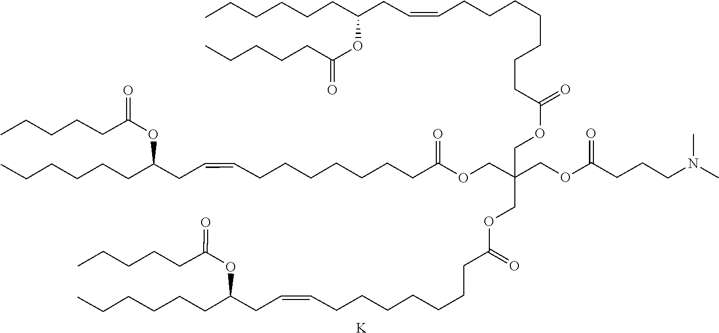 | L1′: 10 carbon chain with C═C cis bond L1″: 1 carbon L1′′′: 6 carbon alkyl L1′′′′: 5 carbon alkyl L2′: 10 carbon chain with C═C cis bond L2″: 1 carbon L2′′′: 6 carbon alkyl L2′′′′: 5 carbon alkyl L3′: 10 carbon chain with C═C cis bond L3″: 1 carbon L3′′′: 6 carbon alkyl L3′′′′: 5 carbon alkyl | N/A | Linker: Formula IIa: W = OC(O) D is absent Xₙ is CH₂ T is absent B = Carbon atom G1, G2 and G3 are present and are each —OC(O)— preceded by CH₂ (Gᵤ) is 1 and R1 and R2 of (CR1R2) are each |
| | | | hydrogen |
| | | | Amine: |
| | | | NMe₂ |
| 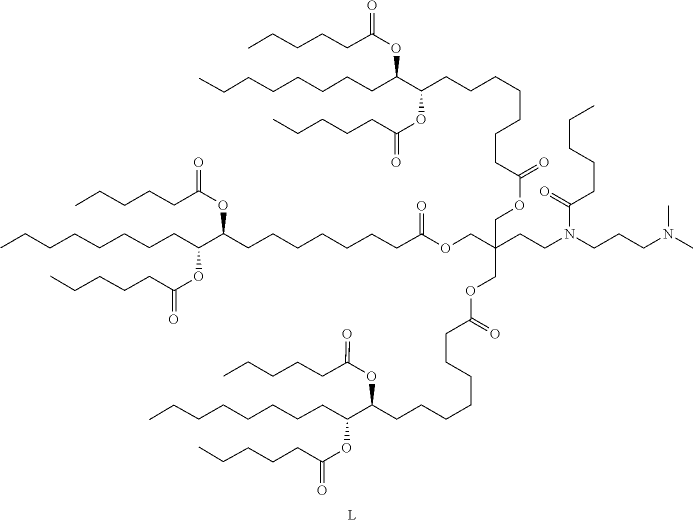 [SMILES: CCCCCCCC[C@@H](OC(=O)CCCCC)[C@H](CCCCCCCC(=O)OCC(CCN(CCCN(C)C)C(=O)CCCCC)(COC(=O)CCCCCCC[C@H](OC(=O)CCCCC)[C@@H](CCCCCCCC)OC(=O)CCCCC)COC(=O)CCCCCCC[C@H](OC(=O)CCCCC)[C@@H](CCCCCCCC)OC(=O)CCCCC)OC(=O)CCCCC] | L1′: 7 carbon alkyl chain L1″: 1 carbon L1′′′: 8 carbon alkyl L1′′′′: 5 carbon alkyl L2′: 7 carbon chain with L2″: 1 carbon L2′′′: 8 carbon alkyl L2′′′′: 5 carbon alkyl L3′: 7 carbon chain with C═C cis bond L3″: 1 carbon L3′′′: 8 carbon alkyl L3′′′′: 5 carbon alkyl | S1′: 5 carbon chain linked to L1′ S2′: 5 carbon alkyl chain linked to L2′′′ S3′: 5 carbon alkyl chain linked to L2′′′ | Linker: Formula IIa: W = —N(R1)C(O)— (R1 = H) D = 5 chain alkyl B = Carbon atom (X)ₙ is 1 and and R1 and R2 of (CR1R2) are each hydrogen G1, G2 and G3 are present and are each —OC(O)— preceded by CH₂ (that is (Gu) is 1 and R1 and R2 of (CR1R2) are each hydrogen) |
| | | | Amine |
| | | | NMe₂ |
| 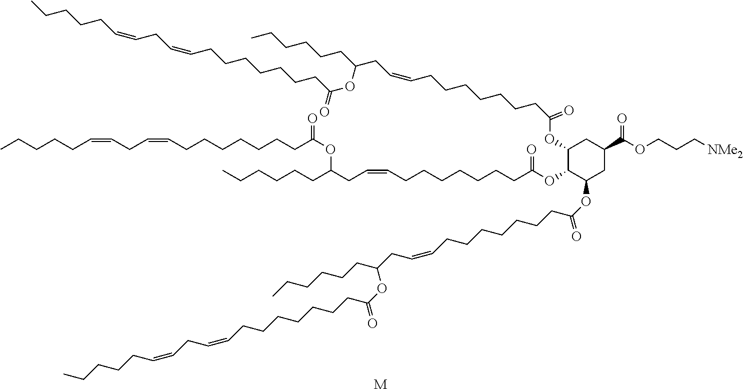 | L1′: 10 carbon chain with C═C cis bond L1″: 1 carbon L1′′′: 6 carbon alkyl L1′′′′: 17 carbon chain with two cis C═C bonds L2′: 10 carbon chain with C═C cis bond L2″: 1 carbon L2′′′: 6 carbon alkyl L2′′′′: 17 carbon chain with two cis C═C bonds L3′: 10 carbon chain with C═C cis bond L3″: 1 carbon L3′′′: 6 carbon alkyl L3′′′′: 17 carbon chain with two cis C═C bonds | N/A | Linker: Derived from quinic acid 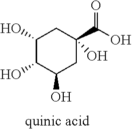 [SMILES: O=C(O)[C@]1(O)C[C@@H](O)[C@@H](O)[C@H](O)C1] Formula IIb: W = —OC(O)— D is not present (X)ₙ is not present (n = 0) and T is not present E—H = cyclohexane ring) |
| | | | (monocyclic 6 |
| | | | carbon ring) |
| | | | G1, G2 and |
| | | | G3 are |
| | | | present |
| | | | and are |
| | | | each |
| | | | —OC(O)— |
| | | | Amine: |
| | | | NMe₂ |
| 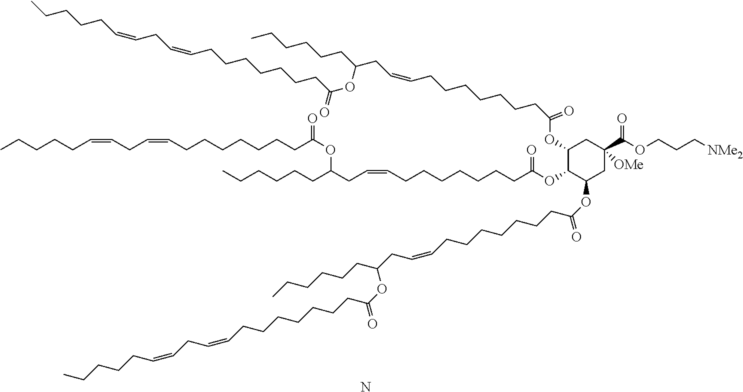 | L1′: 10 carbon chain with C═C cis bond L1″: 1 carbon L1′′′: 6 carbon alkyl L1′′′′: 17 carbon chain with two cis C═C bonds L1′: 10 carbon chain with C═C cis bond L1″: 1 carbon L1′′′: 6 carbon alkyl L1′′′′: 17 carbon chain with two cis C═C bonds | N/A | Linker: Derived from quinic acid 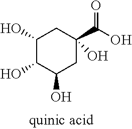 [SMILES: O=C(O)[C@]1(O)C[C@@H](O)[C@@H](O)[C@H](O)C1] Formula IIb: W = —OC(O)— D is not present (X)ₙ is not present (n = 0) and T is not present Amine: NMe₂ |
| | | | E—H = |
| | | | substituted |
| | | | cyclohexane |
| | | | ring |
| | | | (substitution |
| | | | is methoxy, |
| | | | OCH₃) |
| | | | G1, G2 and |
| | | | G3 are |
| | | | present |
| | | | and are |
| | | | each |
| | | | —OC(O)— |
| 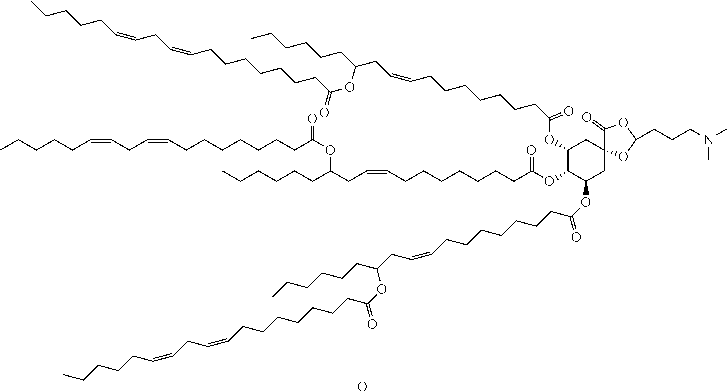 | L1′: 10 carbon chain with C═C cis bond L1″: 1 carbon L1′′′: 6 carbon alkyl L1′′′′: 17 carbon chain with two cis C═C bonds L1′: 10 carbon chain with C═C cis bond L1″: 1 carbon L1′′′: 6 carbon alkyl L1′′′′: 17 carbon chain with two cis C═C bonds | N/A | Linker: Formula IIb: Derived from quinic acid 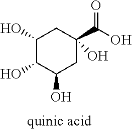 [SMILES: O=C(O)[C@]1(O)C[C@@H](O)[C@@H](O)[C@H](O)C1] W = —OC(O)— D is not present (X)ₙ is not present (n = 0) and T is not present E—H = bicyclic |
| | | | heteroatom |
| | | | G1, G2 and |
| | | | G3 are |
| | | | present |
| | | | and are |
| | | | each |
| | | | —OC(O)— |

An alternative nomenclature for describing the lipidic moiety is Formula IIIc:

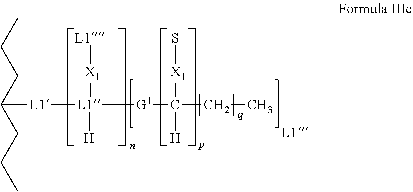

wherein an L backbone is denoted by L1′-L1″-G1-CH—[CH₂]_{q}—CH₃, and wherein the total number of carbon atoms in the L backbone is 10 to 30;
L1′ is a linear hydrocarbon chain and has 2-20, 3-20, 4-20, 5-20, 6-20, 7-20, 8-20, 5-12, 5-10, 5-9, 6-12, 6-10, 6-9, 7-12, 7-10, or 7-9 carbon atoms and 0-3 cis or trans double bonds;
L1″ is a carbon atom;
L1″′ is depicted by G1-CH—CH₂—CH₃;
G¹ is a hydrocarbon chain of 0-4 carbon atoms, optionally having one cis or trans double bond; wherein n is 0 to 4;
wherein p is 1 to 4;
wherein n+p is 1 to 6 or 1 to 4;
q is 0 to 20 or 0 to 10 or 1 to 5;
each X1 is any suitable X1 group described above, or independently selected from an ether, ester and carbamate group;
wherein each S and L1″″ hydrocarbon side chain is independently:
(c) a linear or branched terminating hydrocarbon chain with 0 to 5 cis or trans C═C and 1 to 30 or 2 to 18 carbon atoms and conjugated to one of a respective X1 at any carbon atom in its hydrocarbon chain thereof; or
(d) a branched structure of Formula IIIc,
   wherein the total number of L1″″ and S hydrocarbon chains in Formula IIIc is 1 to 10, 1 to 9, 1 to 8, 1 to 7, 1 to 6, 1 to 5, 1 to 4 or 1 to 3;
   wherein each one of the L1″″ and S hydrocarbon chains in the lipid moiety is optionally substituted with a heteroatom, with the proviso that no more than 4, 3 or 2 heteroatoms are substituted in the hydrocarbon chains.

The X1 ester group can be in any orientation with respect to the location of the carbonyl group as illustrated below:

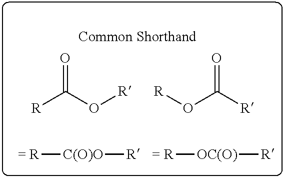

to clarify notation used for describing ester orientation in “G²”

In one embodiment, the total number of carbon atoms in Formula IIIc does not exceed 150, 125, 100, 90, 80, 70, 60 or 50.

In another embodiment, the structure of Formula IIIc is non-conical or flared in shape as determined from a region adjacent to the head group or linker region to a distal carbon atom on the hydrocarbon structure. As noted, such structures facilitate delivery of a cargo molecule when the lipid is formulated in a delivery vehicle.

### Scaffold Carbon Chain

In one embodiment, the backbone hydrocarbon chains of the L1, L2 and/or L3 lipid moieties that provide the scaffold function are derived from a fatty acid with a functional group for linkage to a side chain S. This includes fatty acids substituted with groups having atoms selected from O, N, P and/or S. Such groups facilitate the conjugation of the side chain or chains to a backbone carbon of L1, L2 and/or L3 that make up the scaffold.

For example, L1, L2 and/or L3 may be derived from a hydroxy fatty acid (HFA), which is a fatty acid having an OH group bonded at any position on its carbon chain. Without being limiting, the HFA may be a β-hydroxy fatty, an w-hydroxy fatty acid or any (ω-1)-hydroxy fatty acid, or any other HFA with a reactive functionality at an internal carbon of the carbon backbone. The HFA may be saturated or unsaturated. Two or more hydroxy functional groups can be present on the carbon chain as well.

L1, L2 and/or L3 are alternatively derived from branched fatty acid esters of HFAs known in the art as fatty acid esters of hydroxyl fatty acids (FAHFAs). These fatty acids esters comprise a branched ester linkage between a fatty acid and an HFA. For example, 9-[(9Z)-octadecenoyloxy]octadecanoic acid is a fatty acid ester obtained by condensation of the carboxy group of oleic acid with the hydroxy group of 9-hydroxyoctadecanoic acid.

In alternative embodiments, L1, L2 and/or L3 is derived from a fatty acid amide, which may comprise ethanolamine as the amine component. Yet further, L1, L2 and/or L3 may be derived from fatty acid amines.

The scaffold carbon chain of Formula III may be derived from other fatty acids besides those described above. In addition, it will be appreciated that the fatty acids, in turn, can be derived from their corresponding tri-glycerides.

In Formula III, IIIa and IIIb, L1′, L1″ and L1′″ together form a linear hydrocarbon backbone (referred to herein as a “scaffold carbon chain”). According to Formula IIIc above, the scaffold carbon chain is denoted by L1′-L1″-G¹-CH—[CH₂]_{q}—CH₃, wherein the total number of carbon atoms in the scaffold is 10 to 30.

### Formulations

The cationic or ionizable lipid facilitates the encapsulation of nucleic acid, including but not limited to small interfering RNA, small activating RNA, messenger RNA, microRNA, antisense oligonucleotides, ribozymes, aptamers, plasmids, circular DNA, linear DNA, antagomir, anti-miRNA oligonucleotides and miRNA mimics, and/or gene-editing material. Alternatively or additionally, proteins and amino acids that are negatively charged can be incorporated into the delivery vehicles.

Charged lipids described herein may be used to deliver other charged molecules besides nucleic acids. This includes a wide variety of positively or negatively charged peptides, proteins, polysaccharides or carbohydrates, including both bioactive agents and prodrugs, examples of which are described below.

The cationic or ionizable lipids described herein can be administered in free form with nucleic acid or other negatively or positively charged cargo molecules, or these components can be incorporated into a delivery vehicle. Various delivery systems can be used to prepare pharmaceutical formulations. If the charged lipids and associated charged molecule are in free form, a pharmaceutically acceptable salt or excipient may be included in a pharmaceutical preparation.

The lipids of the present disclosure are particularly amenable to incorporation into nanoparticles, such as liposomes or polymer-based systems comprising lipids or other hydrophobic components, referred to herein as a “lipid nanoparticle” or “LNP”.

For example, in some embodiments, the loading efficiency into a given lipid nanoparticle is 60% to 100%, 70% to 100% or most advantageously 80% to 100%.

In one embodiment, the lipids are loaded into lipid nanoparticles, such as liposomes, by mixing them with lipid formulation components, including vesicle forming lipids and optionally a sterol. As a result, lipid nanoparticles incorporating the ionizable or cationic lipids can be prepared using a wide variety of well described formulation methodologies known to those of skill in the art, including but not limited to extrusion, ethanol injection and in-line mixing. Such methods are described in Maclachlan, I. and P. Cullis, “Diffusible-PEG-lipid Stabilized Plasmid Lipid Particles”, Adv. Genet., 2005. 53PA:157-188; Jeffs, L. B., et al., “A Scalable, Extrusion-free Method for Efficient Liposomal Encapsulation of Plasmid DNA”, Pharm Res, 2005, 22(3):362-72; and Leung, A. K., et al., “Lipid Nanoparticles Containing siRNA Synthesized by Microfluidic Mixing Exhibit an Electron-Dense Nanostructured Core”, The Journal of Physical Chemistry. C, Nanomaterials and Interfaces, 2012, 116(34): 18440-18450, each of which is incorporated herein by reference in its entirety.

Other lipid components that may be included in the lipid nanoparticle besides the charged lipid include vesicle-forming lipids, such as phosphatidylcholine, phosphatidylglycerol, phosphatidylserine, phosphatidylethanolamine, phosphatidic acid, ceramides, or other lipids. Cholesterol may also be included in LNPs to broaden the phase transition temperature. The LNPs may also include a lipid conjugated with a hydrophilic polymer, such as, for example, distearoylphosphatidylethanolamine-PEG. Surface stabilizing functionalities such as hydrophilic polymers may be desirable to reduce clearance of the nanoparticle after administration. The LNP formulations may also include fusogenic lipids that facilitate fusion of the delivery vehicle with a target cell via endocytosis.

Suitable LNPs include, but are not limited to, liposomes prepared by extrusion by known methods or multi-lamellar vesicles (MLVs). The internal space of the liposome may comprise an entrapped agent, such as a drug, or the bilayer or bilayers may comprise such agent partitioned therein.

Another example of a suitable LNP delivery system is a stable nucleic acid-lipid particle, referred to as a SNALP. The SNALP may comprise a nucleic acid associated with the cationic lipid, a non-cationic lipid, and an optional hydrophilic polymer-lipid conjugate, such as a PEGylated lipid and a fusogenic lipid.

A lipid nanoparticle may comprise a lipophilic core. For example, the delivery vehicle can also be a nanoparticle that comprises a lipid core stabilized by a surfactant. Vesicle-forming lipids may be utilized as stabilizers. The lipid nanoparticle in another embodiment is a polymer-lipid hybrid system that comprises a polymer nanoparticle core surrounded by stabilizing lipid.

Nanoparticles may alternatively be prepared from polymers without lipids. Such nanoparticles may comprise a concentrated core of drug that is surrounded by a polymeric shell or may have a solid or a liquid dispersed throughout a polymer matrix.

The lipids described herein can also be incorporated into emulsions, which are drug delivery vehicles that contain oil droplets or an oil core. An emulsion can be lipid-stabilized. For example, an emulsion may comprise an oil filled core stabilized by an emulsifying component such as a monolayer or bilayer of lipids.

The lipids provided herein can also be formulated in micelles. Micelles are self-assembling particles composed of amphipathic lipids or polymeric components that are utilized for the delivery of agents present in the hydrophobic core.

A further class of drug delivery vehicles known to those of skill in which the charged lipid can be formulated is a carbon nanotube.

Various methods for the preparation of the foregoing delivery vehicles and the incorporation of the charged lipids therein are known and may be carried out with ease by those skilled in the art.

Certain lipids encompassed by the disclosure may form part of a carrier-free system. In such embodiments, the lipid associated with a negatively charged molecule could self-assemble into particles. An example is the formation of a lipoplex, which is an association between DNA and a cationic lipid. Such preparations can optionally include a pharmaceutically acceptable salt and/or excipient.

The delivery vehicle incorporating the cationic or ionizable lipid may also include an active agent incorporated in the vehicle, such as an anti-cancer drug or other therapeutic agent, including a pro-drug.

The delivery vehicles may optionally include lipoproteins, such as an apolipoprotein.

### Delivery of Nucleic Acid, Genetic Material, Proteins, Peptides or Other Charged Agents

As discussed, the charged lipid disclosed herein facilitates the incorporation of molecules (referred to herein also as “cargo” or “cargo molecule”) bearing a net negative or positive charge into a delivery vehicle and subsequent delivery to a target cell in vitro or in vivo.

In one embodiment, the molecule is genetic material, such as a nucleic acid. The nucleic acid includes, without limitation, RNA, including small interfering RNA (siRNA), small nuclear RNA (snRNA), micro RNA (miRNA), or DNA such as plasmid DNA or linear DNA. The nucleic acid length can vary and can include nucleic acid of 5-50,000 nucleotides in length. The nucleic acid can be in any form, including single stranded DNA or RNA, double stranded DNA or RNA, or hybrids thereof. Single stranded nucleic acid includes antisense oligonucleotides.

In one particularly advantageous embodiment, the cargo is an siRNA. An siRNA becomes incorporated into endogenous cellular machineries to result in mRNA breakdown, thereby preventing transcription. Since RNA is easily degraded, its incorporation into a delivery vehicle can reduce or prevent such degradation, thereby facilitating delivery to a target site.

Gene editing systems can also be incorporated into delivery vehicles comprising the charged lipid. This includes a Cas9-CRISPR, TALEN and zinc finger nuclease gene editing system. In the case of Cas9-CRISPR, a guide RNA (gRNA), together with a plasmid or mRNA encoding the Cas9 protein may be incorporated into a delivery vehicle comprising the cationic lipid described herein. Optionally, a ribonucleoprotein complex may be incorporated into a delivery vehicle comprising the cationic lipid described herein. Likewise, the disclosure includes embodiments in which genetic material encoding DNA binding and cleavage domains of a zinc finger nuclease or TALEN system are incorporated into a delivery vehicle together with the ionizable or cationic lipid.

The charged lipid can also facilitate the incorporation of proteins and peptides bearing an overall charge into a delivery vehicle. This includes both linear or non-linear peptides. Examples of peptides include bacterial/antibiotic peptides, fungal peptides, invertebrate peptides, amphibian/skin peptides, venom peptides, cancer/anticancer peptides, vaccine peptides, immune/anti-inflammatory peptides, brain peptides, endocrine peptides, ingestive peptides, gastrointestinal peptides, cardiovascular peptides, renal peptides, respiratory peptides, opiate peptides, neurotrophic peptides, and blood-brain peptides. Specific examples of peptides are provided above.

Particular examples of peptides which may associated with the charged lipids described herein are interferons and other macrophage activation factors. This includes lymphokines, muramyl dipeptide (MDP), γ-interferon, α-interferon and β-interferon, and related antiviral and tumoricidal agents; opioid peptides and neuropeptides, which includes enkaphalins, endorphins and dynorphins, and other analgesics; renin inhibitors including for example anti-hypertensive agents; cholecystokinins (CCK analogs) such as CCK, ceruletide and eledoisin, and related cardiovascular-targeting agents and CNS-targeting agents; leukotrienes and prostaglandins, including oxytocin, and other anti-inflammatory, oxytocid and abortifacient compounds; erythropoietin and deratives thereof, as well as related haematinics; LHRH analogs, such as leuprolide, buserelin and nafarelin, and related down-regulators of pituitary receptors; parathyroid hormone and other growth hormone analogs; enzymes, such as Dnase, catalase and alpha-I antitrypsin; immunosuppressants such as cyclosporin; GM-CSF and other immunomodulators; and insulin.

### Administration

In certain embodiments, the charged lipid associated with the nucleic acid or other charged molecule, which is either free or formulated in a drug delivery vehicle, is administered to treat, prevent and/or ameliorate a condition in a patient. In particular, the charged lipid in free form or formulated in a delivery vehicle together with the nucleic acid or other charged material may provide a prophylactic (preventive), ameliorative or a therapeutic benefit. A pharmaceutical composition comprising the charged lipid will be administered at any suitable dosage. In one embodiment, the lipid that is free or formulated in a drug delivery vehicle is administered parentally, i.e., intra-arterially, intravenously, subcutaneously or intramuscularly. In other embodiments, the lipid in free form or formulated in a delivery vehicle described herein may be administered topically. In still further alternative embodiments, the lipid in free form or formulated in a delivery vehicle described herein may be administered orally. In yet a further embodiment, the lipid in free form or formulated in a delivery vehicle is for pulmonary administration by aerosol or powder dispersion.

The compositions described herein may be administered to any subject, including a “patient”, which as used herein includes a human or a non-human subject.

In some embodiments, the lipids described herein are used for the in vitro transfection of cells, including stem cells obtained from a patient and cultured cells. In one embodiment, the cells transfected are stem cells and are administered back to a patient from which they were previously obtained.

The following examples are given for the purpose of illustration only and not by way of limitation on the scope of the invention.

### EXAMPLES

### Materials and Methods

For the organic synthesis reactions described in Example 1, all reagents and solvents were purchased from commercial suppliers and used without further purification unless otherwise stated, except THF (freshly distilled from Na/benzophenone under nitrogen), and Et₃N, DMF and CH₂Cl₂ (freshly distilled from CaH₂ under nitrogen). A USP grade castor oil was purchased at a local pharmacy (Life Brand) and used as received. NMR Chemical shifts are reported in parts per million (ppm) on the δ scale and coupling constants, J, are in hertz (Hz). Spectra are referenced to the signal of the residual solvent. Multiplicities are reported as “s” (singlet), “d” (doublet), “t” (triplet), “q” (quartet), “quint” (quintet), “sept” (septet), “m” (multiplet), and further qualified as “app” (apparent) and “br” (broad).

The lipid nanoparticles (LNPs) were prepared with the neutral lipids, 1,2-distearoyl-sn-glycero-3-phosphocholine (DSPC) and 1,2-dimyristoyl-sn-glycero-3-phosphoethanolamine-N-[methoxy(polyethylene glycol)-2000] (PEG-DMPE), as well as the sterol, cholesterol. DSPC and PEG-DMPE were purchased from Avanti Polar Lipids (Alabaster, Ala.) and cholesterol was obtained from Sigma (St Louis, Mo.).

The LNPs were characterized by measuring particle size and polydispersity (Pdl). The particle size and polydispersity were determined by dynamic light scattering using a Malvern Zetasizer Nano ZS (Malvern, UK). LNP was diluted in appropriate concentrations in PBS. Number-weighted size and distribution data was used in the determination and formulations with Pdl>0.15 were not used for further studies.

Lipid concentrations were determined by measuring total cholesterol using the Cholesterol E enzymatic assay kit from Wako Chemicals USA (Richmond, Va.).

The RNA or antisense oligonucleotide encapsulation efficiency was determined using the Quant-iT Ribogreen RNA or Oligreen ssDNA Assays, respectively (Life Technologies, Burlington, ON). Briefly, LNP was incubated at 37° C. for 10 min in the presence or absence of 1% Triton X-100 (Sigma-Aldrich, St. Louis, Mo.) followed by the addition of the Ribogreen or Oligreen reagent. The fluorescence intensity (Ex/Em: 480/520 nm) was determined and samples treated with Triton X-100 represent total nucleic acid while untreated samples represent unencapsulated nucleic acid.

Quantification of protein/peptide was performed with the use of either the BCA Protein Assay (Pierce) or the CBQCA Protein Quantitation Kit (Invitrogen) according to the manufacturer's instructions.

Apparent acid dissociation constants (pKₐ) of LNP systems were determined according to a procedure described in the literature (Jayaraman, M., et al., Maximizing the potency of siRNA lipid nanoparticles for hepatic gene silencing in vivo. Angewandte Chemie, 2012. 51(34): p. 8529-33). Briefly, 2-(p-toluidino)-6-napthalene sulfonic acid (TNS, Sigma-Aldrich, St. Louis, Mo.) and LNP were diluted in buffer (10 mM HEPES, 10 mM MES and 10 mM ammonium acetate) with pH ranges from 2.5 to 11. Final concentration of TNS or total lipid was 6 μM. Samples were then mixed and fluorescence intensity was measured (Ex/Em: 321/445 nm) using a Perkin Elmer LS55. A sigmoidal best fit analysis was applied and the pKₐ was measured as the pH at half-maximal fluorescence intensity.

### Example 1: Synthesis of Ionizable Lipids

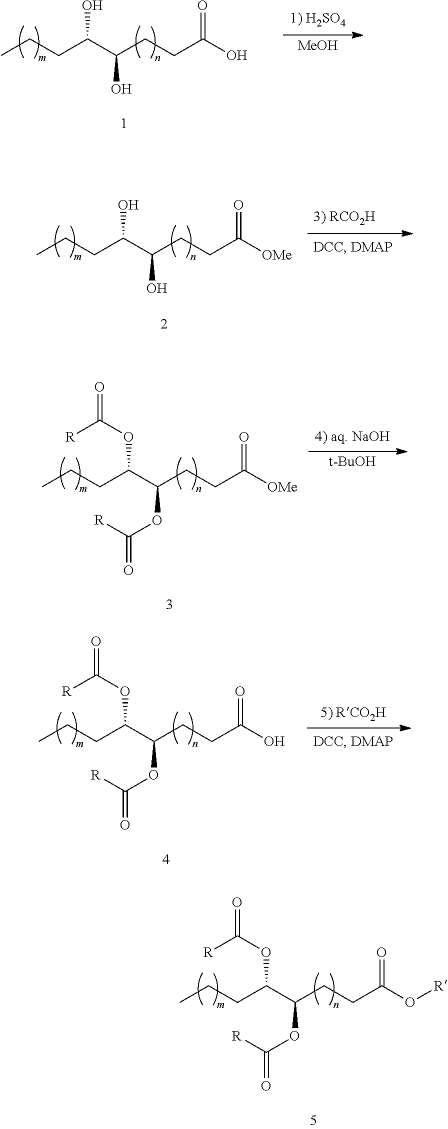

### General Procedure A—Esterification of Hydroxy Fatty Acids

The hydroxylated fatty acid (1.00 equiv.) was suspended in MeOH (0.4-0.5 M) in a round bottom flask equipped with a condenser. Concentrated sulfuric acid (0.05 equiv.) was added to the above mixture and the resultant heated at reflux, which became homogeneous in 5-15 min. After 16 h, residual MeOH was removed on a rotary evaporator and the remaining residue was partitioned between ethyl acetate (EtOAc) and saturated aqueous NaHCO₃. The aqueous layer was extracted twice with EtOAc and the combined organic layers were washed once with water, brine, dried over Na₂SO₄, gravity filtered and concentrated on a rotary evaporator to afford white powder, which was used without further purification.

### General Procedure B—Acylation of Hydroxy Fatty Acid Esters

N,N′-Dicyclohexylcarbodiimide (DCC; 1.00 equiv. per hydroxyl+additional 0.10 equiv.) was added to an ice-cold CH₂Cl₂ (0.3 M) solution of the desired carboxylic acid (1.00 equiv. per hydroxyl+additional 0.10 equiv.) in a round bottom flask under argon. Subsequently, the ice bath was removed and the resultant mixture stirred for 15 min. The reaction mixture was cooled again in an ice bath and solid hydroxy fatty acid (1.00 equiv.) was added thereto, followed by the addition of 4-dimethylaminopyridine (DMAP; 1.00 equiv. per hydroxyl+additional 0.50 equiv.). The reaction mixture was allowed to warm to room temperature over 16 h, then diluted with hexanes, stirred for 10 min and subsequently filtered through a pad of Celite®. The filtrate was concentrated on a rotary evaporator to yield a crude mixture from which the desired acylated material was purified by flash column chromatography.

### General Procedure C—Saponification of Peracylated Fatty Acid Methyl Esters

Aqueous NaOH (2.0 M, 1.00 equiv.) was added to a room temperature t-BuOH (0.3 M) solution of acylated fatty acid methyl ester (1.10 equiv.) in a round bottom flask under argon. After stirring for 16 h, the reaction mixture was acidified to pH 2 by addition of aqueous HCl (2.0 M) and extracted three times with hexanes. The combined organic extracts were washed with brine, dried over Na₂SO₄ and concentrated on a rotary evaporator to afford the crude as a colourless oil. The crude was purified by flash column chromatography to afford the desired fatty acids.

General Procedure D—Esterification of Peracylated Fatty Acids with Aminoalcohols

DCC (1.10 equiv.) was added to an ice-cold CH₂Cl₂ (0.2 M) solution of the fatty acid (1.00 equiv.) in a round bottom flask under argon. The ice bath was removed and the resultant stirred for 15 min. The reaction mixture was cooled again in an ice bath, neat aminoalcohol (1.20-2.00 equiv.) was added, followed by DMAP (1.20 equiv.), and the reaction mixture was allowed to warm to room temperature over 16 h. The reaction mixture was diluted with Et₂O, stirred for 10 min and subsequently filtered through a pad of Celite®. The filtrate was concentrated on a rotary evaporator to yield a crude oil, which was purified by flash column chromatography to afford the desired peracylated aminolipids.

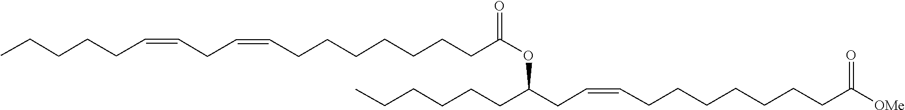 [SMILES: CCCCC/C=C\C/C=C\CCCCCCCC(=O)O[C@@H](C/C=C\CCCCCCCC(=O)OC)CCCCCC]

### Methyl (12R)-linoleoxyoleate

According to General Procedure B, methyl ricinoleate (500 mg, 1.60 mmol), linoleic acid (538 mg, 1.92 mmol, 1.20 equiv.), DCC (396 mg, 1.20 mmol, 2.20 equiv.) and DMAP (293 mg, 2.40 mmol, 1.50 equiv.) in CH₂Cl₂ (5 mL) afforded the title compound (875 mg, 93% yield) as a clear, colourless oil.

¹H (300 MHz, CDCl₃): 5.55-5.28 (m, 6H), 4.90 (quint, J=6.2 Hz, 1H), 3.69 (s, 3H), 2.79 (t, J=5.8 Hz, 2H), 2.40-2.21 (m, 6H), 2.16-1.93 (m, 6H), 1.72-1.46 (m, 8H), 1.46-1.18 (m, 32H), 1.00-0.80 (m, 6H).

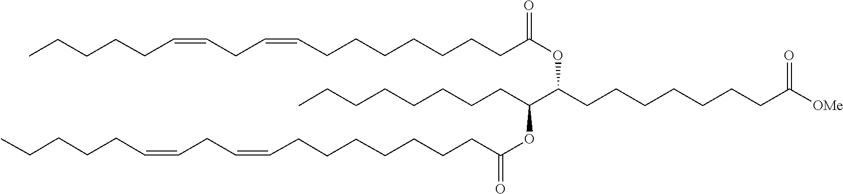 [SMILES: CCCCC/C=C\C/C=C\CCCCCCCC(=O)O[C@@H](CCCCCCCC)[C@@H](CCCCCCCC(=O)OC)OC(=O)CCCCCCC/C=C\C/C=C\CCCCC]

### Methyl (±)-syn-9,10-dilinoleoxystearate

According to General Procedure B, the dihydroxy stearate (1.32 g, 4.00 mmol), linoleic acid (2.47 g, 8.80 mmol, 2.20 equiv.), DCC (1.82 g, 8.80 mmol, 2.20 equiv.) and DMAP (1.22 g, 10.0 mmol, 2.50 equiv.) in CH₂Cl₂ (10 mL) afforded the title compound (2.64 g, 77% yield) as a clear, colourless oil.

¹H (300 MHz, CDCl₃): 5.47-5.28 (m, 8H), 5.04-4.94 (m, 2H), 3.68 (s, 3H), 2.79 (t, J=5.8 Hz, 4H), 2.35-2.25 (m, 6H), 2.11-2.00 (m, 8H), 1.69-1.47 (m, 10H), 1.44-1.16 (m, 48H), 0.97-0.84 (m, 9H).

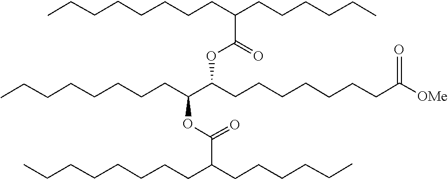 [SMILES: CCCCCCCCC(CCCCCC)C(=O)O[C@@H](CCCCCCCC)[C@@H](CCCCCCCC(=O)OC)OC(=O)C(CCCCCC)CCCCCCCC]

### Methyl (±)-syn-9,10-bis(2-hexyldecanoyloxy)stearate

According to General Procedure B, the dihydroxy stearate (2.31 g, 7.00 mmol), (±)-2-hexyldecanoic acid (3.77 g, 14.7 mmol, 2.10 equiv.), DCC (3.03 g, 14.7 mmol, 2.10 equiv.) and DMAP (2.14 g, 17.5 mmol, 2.50 equiv.) in CH₂Cl₂ (18 mL) afforded the title compound (4.20 g, 74% yield) as a clear, colourless oil.

¹H (300 MHz, CDCl₃): 5.06-4.96 (m, 2H), 3.68 (s, 3H), 2.31 (t, J=7.6 Hz, 2H), 1.69-1.50 (m, 10H), 1.50-1.16 (m, 64H), 0.94-0.84 (m, 15H).

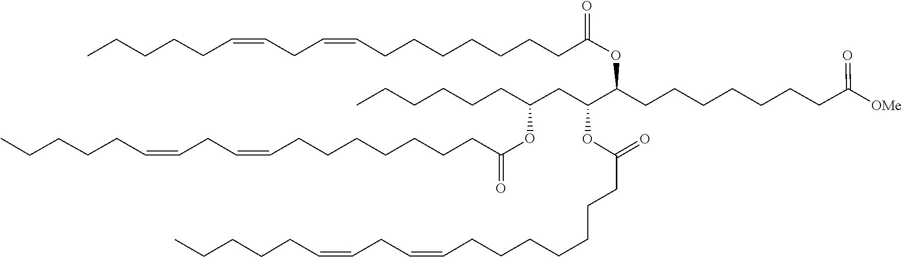 [SMILES: CCCCC/C=C\C/C=C\CCCCCCCC(=O)O[C@H](CCCCCC)C[C@@H](OC(=O)CCCCCCC/C=C\C/C=C\CCCCC)[C@H](CCCCCCCC(=O)OC)OC(=O)CCCCCCC/C=C\C/C=C\CCCCC]

### Methyl (syn-9,10,12R)-trilinoleoxystearate

According to General Procedure B, the trihydroxy stearate (1.04 g, 3.00 mmol), linoleic acid (2.61 g, 9.30 mmol, 3.10 equiv.), DCC (1.92 g, 9.30 mmol, 3.10 equiv.) and DMAP (1.28 g, 10.5 mmol, 3.50 equiv.) in CH₂Cl₂ (10 mL) afforded the title compound (2.33 g, 68% yield) as a clear, colourless oil.

¹H (300 MHz, CDCl₃): 5.49-5.26 (m, 12H), 5.14-4.84 (m, 3H), 3.68 (s, 3H), 2.79 (br t, J=6.0 Hz, 6H), 2.37-2.21 (m, 8H), 2.14-1.99 (m, 12H), 1.92-1.47 (m, 16H), 1.47-1.16 (m, 56H), 0.96-0.84 (m, 12H).

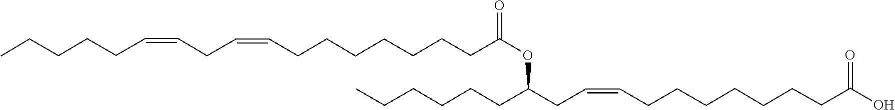 [SMILES: CCCCC/C=C\C/C=C\CCCCCCCC(=O)O[C@@H](C/C=C\CCCCCCCC(=O)O)CCCCCC]

### (12R)-Linoleoxyoleic acid

According to General Procedure C, the acyl methyl ester (5.97 g, 10.4 mmol, 1.10 equiv.), aqueous NaOH (2.0 M, 4.70 mL, 1.00 equiv.) and t-BuOH (26 mL) afforded, after flash column chromatography (SiO₂, 95:5:0→90:10:0→85:12:3 hexanes/EtOAc/MeOH), the title compound (4.48 g, 85% yield) as a clear, colourless oil.

¹H (300 MHz, CDCl₃): 5.55-5.28 (m, 6H), 4.90 (quint, J=6.2 Hz, 1H), 2.79 (t, J=6.0 Hz, 2H), 2.43-2.21 (m, 6H), 2.14-1.96 (m, 6H), 1.73-1.47 (m, 6H), 1.46-1.18 (m, 30H), 0.99-0.81 (m, 6H).

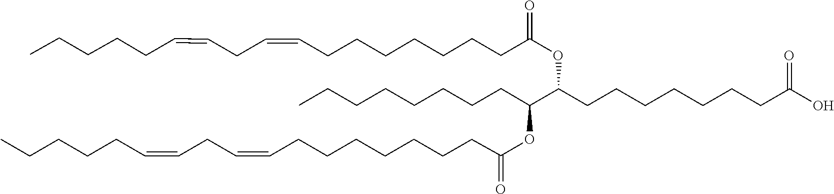 [SMILES: CCCCC/C=C\C/C=C\CCCCCCCC(=O)O[C@@H](CCCCCCCC)[C@@H](CCCCCCCC(=O)O)OC(=O)CCCCCCC/C=C\C/C=C\CCCCC]

### (±)-syn-9,10-Dilinoleoxystearic Acid (INT-A008)

According to General Procedure C, the diacyl methyl ester (1.88 g, 2.20 mmol, 1.10 equiv.), aqueous NaOH (2.0 M, 1.00 mL) and t-BuOH (7 mL) afforded, after flash column chromatography (SiO₂, 95:5:0→90:10:0→85:12:3 hexanes/EtOAc/MeOH), INT-A008 (1.50 g, 89% yield) as a clear, colourless oil.

¹H (300 MHz, CDCl₃): 5.47-5.28 (m, 8H), 5.04-4.94 (m, 2H), 2.79 (br t, J=6.0 Hz, 4H), 2.36 (t, J=7.0 Hz, 2H), 2.30 (t, J=7.5 Hz, 4H), 2.12-1.98 (m, 8H), 1.72-1.46 (m, 10H), 1.44-1.16 (m, 48H), 0.97-0.84 (m, 9H).

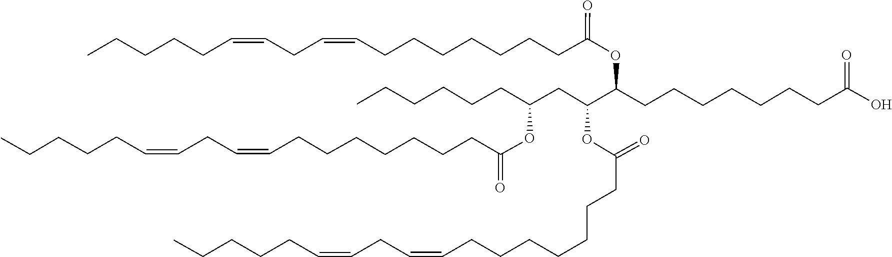 [SMILES: CCCCC/C=C\C/C=C\CCCCCCCC(=O)O[C@H](CCCCCC)C[C@@H](OC(=O)CCCCCCC/C=C\C/C=C\CCCCC)[C@H](CCCCCCCC(=O)O)OC(=O)CCCCCCC/C=C\C/C=C\CCCCC]

### (syn-9,10,12R)-Trilinoleoxystearic Acid

According to General Procedure C, the triacyl methyl ester (2.41 g, 2.12 mmol, 1.05 equiv.), aqueous NaOH (2.0 M, 1.01 mL) and t-BuOH (7 mL) afforded, after flash column chromatography (SiO₂, 90:10:0→80:17:3 hexanes/EtOAc/MeOH), the triacylated fatty acid (1.37 g, 60% yield) as a clear, colourless oil.

¹H (300 MHz, CDCl₃): 5.47-5.26 (m, 12H), 5.14-4.84 (m, 3H), 2.79 (br t, J=6.0 Hz, 6H), 2.41-2.20 (m, 8H), 2.14-1.99 (m, 12H), 1.92-1.47 (m, 16H), 1.47-1.16 (m, 56H), 0.96-0.84 (m, 12H).

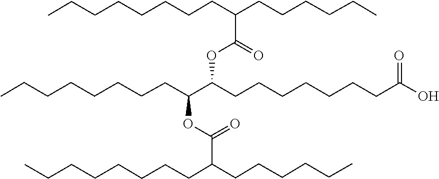 [SMILES: CCCCCCCCC(CCCCCC)C(=O)O[C@@H](CCCCCCCC)[C@@H](CCCCCCCC(=O)O)OC(=O)C(CCCCCC)CCCCCCCC]

### (±)-syn-9,10-Bis(2-hexyldecanoyloxy)stearic Acid (INT-A009)

According to General Procedure C, the diacyl methyl ester (4.20 g, 5.21 mmol, 1.05 equiv.), aqueous NaOH (2.0 M, 2.48 mL) and t-BuOH (7 mL) afforded, after flash column chromatography (SiO₂, 95:5:0→90:10:0→85:12:3 hexanes/EtOAc/MeOH), INT-A009 (3.12 g, 79% yield) as a clear, colourless oil.

¹H (300 MHz, CDCl₃): 5.09-4.93 (m, 2H), 2.41-2.23 (m, 4H), 1.71-1.51 (m, 10H), 1.51-1.15 (m, 64H), 0.95-0.82 (m, 15H).

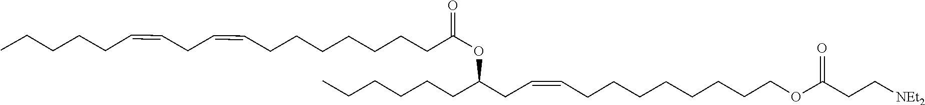

### 3-(Dimethylamino)propyl (12R)-linoleoxyoleate (INT-A005)

According to General Procedure D, the carboxylic acid (561 mg, 1.00 mmol), aminoalcohol (0.18 mL, 1.20 mmol, 1.20 equiv.), DCC (227 mg, 1.10 mmol, 1.10 equiv.) and DMAP (147 mg, 1.20 mmol, 1.20 equiv.) in CH₂Cl₂ (3.5 mL), followed by flash column chromatography (SiO₂, 88:10:268:30:2 hexanes/EtOAc/Et₃N), afforded INT-A005 (639 mg, 95% yield) as a clear, colourless oil.

¹H (300 MHz, CDCl₃): 5.54-5.23 (m, 6H), 4.89 (quint, J=6.0 Hz, 1H), 4.12 (t, J=6.0 Hz, 2H), 2.78 (br t, J=6.0 Hz, 2H), 2.58-2.45 (m, 6H), 2.29 (br q, J=6.0 Hz, 6H), 2.12-1.96 (m, 6H), 1.83-1.72 (m, 2H), 1.69-1.47 (m, 6H), 1.44-1.18 (m, 30H), 1.02 (t, J=7.1 Hz, 6H), 0.94-0.84 (m, 6H).

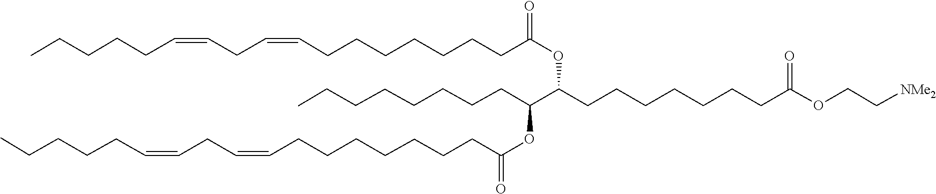 [SMILES: CCCCC/C=C\C/C=C\CCCCCCCC(=O)O[C@@H](CCCCCCCC)[C@@H](CCCCCCCC(=O)OCCN(C)C)OC(=O)CCCCCCC/C=C\C/C=C\CCCCC]

### 2-(Dimethylamino)ethyl (±)-syn-9,10-dilinoleoxystearate (INT-A001)

According to General Procedure D, the carboxylic acid (336 mg, 0.40 mmol), aminoalcohol (0.12 mL, 1.20 mmol, 3.00 equiv.), DCC (91 mg, 0.44 mmol, 1.10 equiv.) and DMAP (59 mg, 0.48 mmol, 1.20 equiv.) in CH₂Cl₂ (2 mL), followed by flash column chromatography (SiO₂, 88:20:268:30:248:50:2 hexanes/EtOAc/Et₃N), afforded INT-A001 (255 mg, 70% yield) as a clear, colourless oil.

¹H (300 MHz, CDCl₃): 5.47-5.27 (m, 8H), 5.04-4.94 (m, 2H), 4.18 (t, J=5.9 Hz, 2H), 2.79 (br t, J=5.90 Hz, 4H), 2.57 (t, J=5.8 Hz, 2H), 2.39-2.24 (m, 9H), 2.30 (s, 3H), 2.13-1.99 (m, 8H), 1.70-1.46 (m, 10H), 1.44-1.16 (m, 48H), 0.97-0.84 (m, 9H).

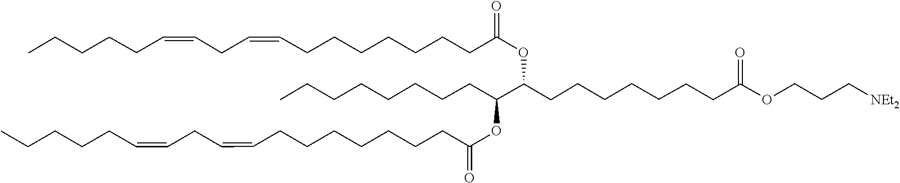

### 3-(Diethylamino)propyl (±)-syn-9,10-dilinoleoxystearate (INT-A002)

According to General Procedure D, the carboxylic acid (336 mg, 0.40 mmol), aminoalcohol (0.12 mL, 0.80 mmol, 2.00 equiv.), DCC (91 mg, 0.44 mmol, 1.10 equiv.) and DMAP (59 mg, 0.48 mmol, 1.20 equiv.) in CH₂Cl₂ (2 mL), followed by flash column chromatography (SiO₂, 78:20:268:30:2 hexanes/EtOAc/Et₃N), afforded INT-A002 (272 mg, 71% yield) as a clear, colourless oil.

¹H (300 MHz, CDCl₃): 5.47-5.27 (m, 8H), 5.04-4.94 (m, 2H), 4.12 (t, J=6.5 Hz, 2H), 2.79 (br t, J=5.9 Hz, 4H), 2.59-2.46 (m, 6H), 2.30 (t, J=7.5 Hz, 6H), 2.12-1.99 (m, 8H), 1.78 (quint, J=6.9 Hz, 2H), 1.70-1.46 (m, 10H), 1.44-1.16 (m, 48H), 1.03 (t, J=7.1 Hz, 6H), 0.97-0.84 (m, 9H).

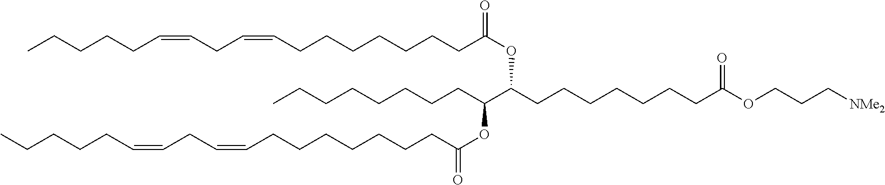 [SMILES: CCCCC/C=C\C/C=C\CCCCCCCC(=O)O[C@@H](CCCCCCCC)[C@@H](CCCCCCCC(=O)OCCCN(C)C)OC(=O)CCCCCCC/C=C\C/C=C\CCCCC]

### 3-(Dimethylamino)propyl (±)-syn-9,10-dilinoleoxystearate (INT-A003)

According to General Procedure D, the carboxylic acid (421 mg, 0.50 mmol), aminoalcohol (88 μL, 0.75 mmol, 1.50 equiv.), DCC (113 mg, 0.55 mmol, 1.10 equiv.) and DMAP (73 mg, 0.60 mmol, 1.20 equiv.) in CH₂Cl₂ (3 mL), followed by flash column chromatography (SiO₂, 78:20:268:30:2 hexanes/EtOAc/Et₃N), afforded INT-A003 (323 mg, 70% yield) as a clear, colourless oil.

¹H (300 MHz, CDCl₃): 5.47-5.27 (m, 8H), 5.04-4.94 (m, 2H), 4.13 (t, J=6.60 Hz, 2H), 2.79 (br t, J=5.90 Hz, 4H), 2.39-2.25 (m, 8H), 2.24 (s, 3H), 2.13-1.99 (m, 8H), 1.81 (quint, J=6.8 Hz, 2H), 1.70-1.46 (m, 10H), 1.44-1.16 (m, 48H), 0.97-0.84 (m, 9H).

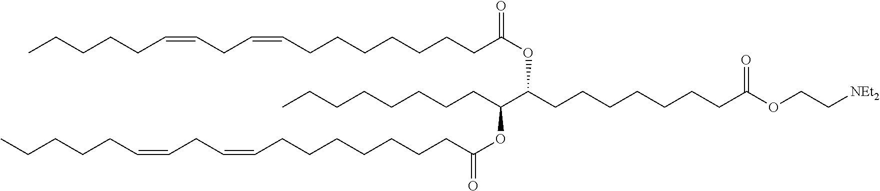

### 2-(Diethylamino)ethyl (±)-syn-9,10-dilinoleoxystearate (INT-A004)

According to General Procedure D, the carboxylic acid (421 mg, 0.50 mmol), aminoalcohol (80 μL, 0.60 mmol, 1.20 equiv.), DCC (113 mg, 0.55 mmol, 1.10 equiv.) and DMAP (73 mg, 0.60 mmol, 1.20 equiv.) in CH₂Cl₂ (3 mL), followed by flash column chromatography (SiO₂, 78:20:268:30:2 hexanes/EtOAc/Et₃N), afforded INT-A004 (406 mg, 86% yield) as a clear, colourless oil.

¹H (300 MHz, CDCl₃): 5.47-5.27 (m, 8H), 5.05-4.93 (m, 2H), 4.15 (t, J=6.3 Hz, 2H), 2.78 (br t, J=5.9 Hz, 4H), 2.70 (t, J=6.1 Hz, 2H), 2.59 (q, J=7.2 Hz, 4H), 2.36-2.23 (m, 6H), 2.12-1.99 (m, 8H), 1.69-1.45 (m, 10H), 1.44-1.16 (m, 48H), 1.05 (t, J=7.1 Hz, 6H), 0.97-0.84 (m, 9H).

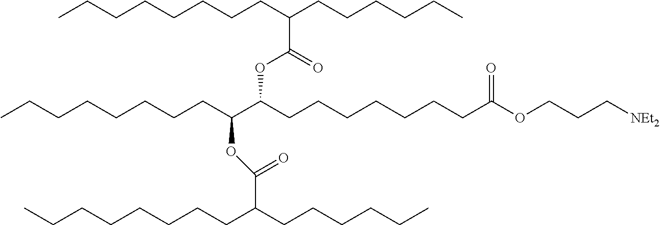

### 3-(Diethylamino)propyl (±)-syn-9,10-bis(2-hexyldecanoyloxy)stearate (INT-A007)

According to General Procedure D, the carboxylic acid (595 mg, 0.75 mmol), aminoalcohol (0.13 mL, 0.90 mmol, 1.20 equiv.), DCC (170 mg, 0.82 mmol, 1.10 equiv.) and DMAP (110 mg, 0.90 mmol, 1.20 equiv.) in CH₂Cl₂ (4 mL), followed by flash column chromatography (SiO₂, 85:15:080:15:575:20:5 hexanes/EtOAc/MeOH), afforded INT-A007 (577 mg, 85% yield) as a clear, colourless oil.

¹H (300 MHz, CDCl₃): 5.06-4.96 (m, 2H), 4.12 (t, J=6.5 Hz, 2H), 2.60-2.46 (m, 6H), 2.37-2.23 (m, 5H), 1.79 (quint, J=7.2 Hz, 2H), 1.70-1.50 (m, 10H), 1.50-1.16 (m, 64H), 1.04 (t, J=7.1 Hz, 6H) 0.94-0.84 (m, 15H).

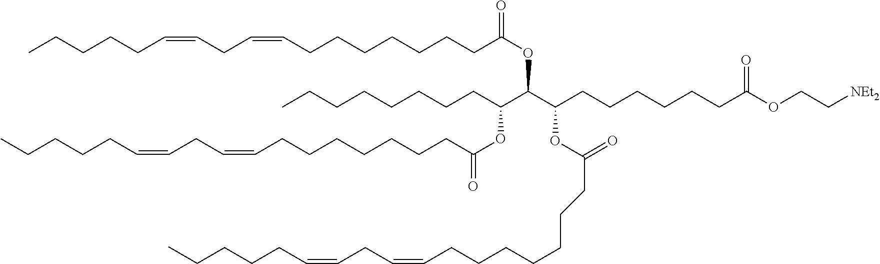

### 3-(Diethylamino)propyl(±)syn-9,10,12R)-trilinoleoxystearate (INT-A006)

According to General Procedure D, the carboxylic acid (560 mg, 0.50 mmol), aminoalcohol (89 μL, 0.0.60 mmol, 1.20 equiv.), DCC (113 mg, 0.55 mmol, 1.10 equiv.) and DMAP (73 mg, 0.60 mmol, 1.20 equiv.) in CH₂Cl₂ (3 mL), followed by flash column chromatography (SiO₂, 85:15:080:15:575:20:5 hexanes/EtOAc/MeOH), afforded INT-A006 (417 mg, 68% yield) as a clear, colourless oil.

¹H (300 MHz, CDCl₃): 5.49-5.26 (m, 12H), 5.12-4.84 (m, 3H), 4.12 (t, J=6.5 Hz, 2H), 2.59-2.45 (m, 6H), 2.36-2.21 (m, 8H), 2.14-1.99 (m, 12H), 1.92-1.47 (m, 18H), 1.47-1.16 (m, 56H), 1.03 (t, J=7.1 Hz, 6H), 0.96-0.84 (m, 12H).

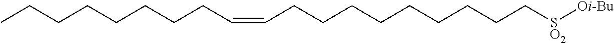

### Isobutyl (10Z)-nonadecenesulfonate

In adaptation of a procedure (M. Xie, T. S. Widlanski, Tetrahedron Lett. 1996, 37, 4443), n-BuLi solution (6.52 mL of 1.15 M in hexanes, 7.50 mmol, 1.50 equiv.) was added to a −78° C. 9:1 THF/DMPU (15 mL) solution of isobutyl methanesulfonate (1.22 g, 8.00 mmol, 1.60 equiv.) in a round bottom flask under argon and the resultant allowed to stir for 30 min. While still at −78° C., a THF (2 mL) solution of oleyl iodide (1.89 g, 5.00 mmol, 1.00 equiv.) was added to the above solution and the reaction mixture was allowed to warm up over 16 h. The reaction mixture was quenched with aqueous 10% citric acid, extracted with Et₂O (2×10 mL) and the combined organics washed with water (1×10 mL), brine (1×10 mL), then dried over Na₂SO₄ and concentrated on a rotary evaporator. The crude residue was purified by flash column chromatography (98:2→95:5 hexanes/EtOAc) to afford the alkylated sulfonate (1.19 g, 46% yield) as a clear, colourless oil.

¹H (300 MHz, CDCl₃): 5.46-5.27 (m, 2H), 4.00 (d, J=6.6 Hz, 2H), 3.14-3.05 (m, 2H), 2.11-1.94 (m, 5H), 1.94-1.80 (m, 2H), 1.51-1.18 (m, 26H), 1.00 (t, J=6.8 Hz, 6H), 0.90 (br t, J=6.6 Hz, 3H).

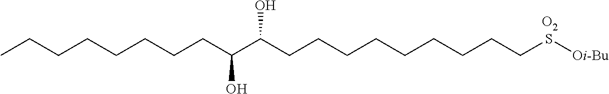

### Isobutyl (±)-syn-10,11-dihydroxynonadecanesulfonate

OSO₄ solution (0.11 mL of 4% in water, 0.02 mmol, 0.01 equiv.) was added to a room temperature 4:1 Me₂CO/H₂O (5.5 mL) solution of Isobutyl (10Z)-nonadecenesulfonate (709 mg, 1.76 mmol) and NMO (0.54 mL of 50% in water, 2.64 mmol, 1.50 equiv.) in a round bottom flask under argon. After stirring for 16 h, saturated aqueous NaHSO₃ was added and the reaction mixture allowed to stir for 1 h, at which point it was extracted with EtOAc (3×10 mL) and the combined organics were washed with water (1×10 mL), brine (1×10 mL), dried over Na₂SO₄ and concentrated on a rotary evaporator to afford the diol (768 mg, quantitative yield) as a white solid that was used without further purification.

¹H (300 MHz, CDCl₃): 4.00 (d, J=6.6 Hz, 2H), 3.67-3.65 (m, 2H), 3.14-3.05 (m, 2H), 2.05 (sept, J=6.7 Hz, 1H), 1.95-1.78 (m, 4H), 1.58-1.19 (m, 28H), 1.00 (t, J=6.8 Hz, 6H), 0.90 (br t, J=6.6 Hz, 3H).

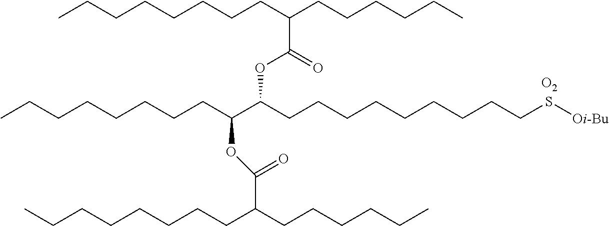

Isobutyl (±)-syn-10,11-bis(2-hexyldecanoyloxy)nonadecanesulfonate

Solid DCC (388 mg, 1.88 mmol, 2.10 equiv.) was added to an ice-cold CH₂Cl₂ (4.5 mL) solution of (±)-2-hexyldecanoic acid (482 mg, 1.88 mmol, 2.10 equiv.) in a round bottom flask under argon. Subsequently, the ice bath was removed and the resultant mixture stirred for 15 min. The reaction mixture was cooled again in an ice bath and solid dihydroxy sulfonate (391 mg, 0.90 mmol, 1.00 equiv.) was added thereto, followed by DMAP (273 mg, 2.23 mmol, 2.50 equiv.). The reaction mixture was allowed to warm to room temperature over 16 h, then diluted with Et₂O, stirred for 10 min and subsequently filtered through a pad of Celite®. The filtrate was concentrated on a rotary evaporator, then purified by flash column chromatography (SiO₂, 95:590:10 hexanes/EtOAc) to afford the diacylated sulfonate (574 mg, 70% yield) as a clear, colourless oil.

¹H (300 MHz, CDCl₃): 5.08-4.93 (m, 2H), 4.00 (d, J=6.6 Hz, 2H), 3.67-3.65 (m, 2H), 3.14-3.05 (m, 2H), 2.38-2.24 (m, 2H), 2.05 (sept, J=6.7 Hz, 1H), 1.95-1.80 (m, 2H), 1.70-1.15 (m, 74H), 1.00 (t, J=6.8 Hz, 6H), 0.96-0.83 (m, 15H).

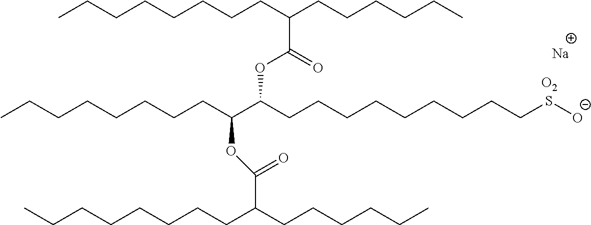

### Sodium (±)-syn-10,11-bis(2-hexyldecanoyloxy)nonadecanesulfonate (INT-A012)

NaI (82 mg, 0.55 mmol, 2.00 equiv.) was added to a room temperature Me₂CO (0.9 mL) solution of diacylated isobutyl sulfonate (250 mg, 0.27 mmol) in a sealed glass tube with a Teflon® screw cap and under argon. The reaction mixture was then heated at reflux for 24 h, at which point it was diluted with Me₂CO (3 mL), cooled on ice for 2-3 h and the white precipitate was collected and washed with ice-cold Me₂CO to afford INT-A012 as a white powder (200 mg, 84% yield)

¹H (300 MHz, CDCl₃): 5.10-4.93 (m, 2H), 2.90 (app br s, 2H), 2.46-2.22 (m, 4H), 1.70-1.15 (m, 76H), 0.96-0.83 (m, 15H).

### Example 2: Formulation of Lipid Nanoparticles Containing Nucleic Acids into Lipid Nanoparticles (LNPs)

The lipids, INT-A001, INT-A002, INT-A003, INT-A004, INT-A005, INT-A006 and INT-A007, synthesized as described in Example 1 were formulated in lipid nanoparticles together with a nucleic acid. The nucleic acid for incorporation of the LNP as an example cargo was siRNA against Factor VII, which is a protein involved in blood coagulation. Factor VII levels can be easily measured in blood plasma by a chromogenic assay and thus represents a convenient model for determining siRNA-mediated downregulation of this factor.

Physiochemical parameters, including apparent pKₐ, particle diameter, polydispersity (PDI) and encapsulation efficiency of the nanoparticles with incorporated cationic lipid and siRNA against Factor VII were measured and reported below. The results below show that the LNPs were suitable for the encapsulation and delivery of the nucleic acid.

To prepare the LNPs, ionizable lipid, DSPC, cholesterol and PEG-DMPE were dissolved in ethanol. The siRNA was dissolved in pH 4.0-6.2 buffer composed of 10-50 mM acetate, succinate or citrate. In select cases, 10-50 mM 4-(2-Hydroxyethyl)piperazine-1-ethanesulfonic acid (HEPES) or 2-(N-Morpholino)ethanesulfonic acid (MES) buffer was used. LNP were prepared by rapidly mixing the lipid components in ethanol (in molar ratio of 50/10/38.5/1.5 of DSPC/chol/PEG-DMPE) with nucleic acids in aqueous buffer at a volumetric flow rate ratio of 1:3 (ethanol to aqueous, combined flow rate >12 mL/min) at room temperature. Typically, siRNA/lipids ratios were targeted for 0.056 wt/μmol. The product was then dialyzed against 1× phosphate-buffered saline (PBS) at pH 7.4 for 24 hours to remove residual ethanol and to raise the pH. PBS was refreshed after 4 hours.

As shown in Table 2 below, the INT-A001, INT-A002, INT-A003, INT-A004, INT-A005, INT-A006 and INT-A007 ionizable lipids facilitated the incorporation of FVII siRNA at high encapsulation efficiency and low polydispersity, both of which are desirable physiochemical properties for drug delivery systems. The apparent pKₐ values for these ionizable lipids were also measured as certain ionizable lipids with apparent pKₐ values between 6 to 7 were previously reported to be active in mediating gene silencing (Semple, S. C., et al., Rational design of cationic lipids for siRNA delivery. Nat Biotechnol, 2010, 28(2): p. 172-6 and Jayaraman, M., et al., Maximizing the potency of siRNA lipid nanoparticles for hepatic gene silencing in vivo. Angewandte Chemie, 2012, 51(34): p. 8529-33).

It was determined that INT-A001, INT-A002, INT-A003, INT-A004, INT-A005, INT-A006 and INT-A007 exhibit pKₐ values within the appropriate range of 6 to 7 (Table 2).

TABLE 2
Physiochemical parameters of LNP containing ionizable lipids and siRNA

| Ionizable | Apparent | Particle Diameter | Polydispersity | Encapsulation |
|---|---|---|---|---|
| Lipid ID | pKₐ | (nm) | index (PDI) | Efficiency (%) |
| INT-A001 | 6.3 | 50 | 0.059 | 89 |
| INT-A002 | 6.8 | 52 | 0.063 | 97 |
| INT-A003 | 7.0 | 55 | 0.043 | 92 |
| INT-A004 | 5.8 | 58 | 0.024 | 61 |
| INT-A005 | 7.0 | 49 | 0.035 | 87 |
| INT-A006 | 6.7 | 64 | 0.020 | 83 |
| INT-A007 | 6.9 | 59 | 0.021 | 84 |

Cumulatively, this data demonstrates the suitability of these novel ionizable lipids for the encapsulation of nucleic acid in LNPs.

### Example 3: Determination of LNP Activity in Cultured 22Rv1 Cells

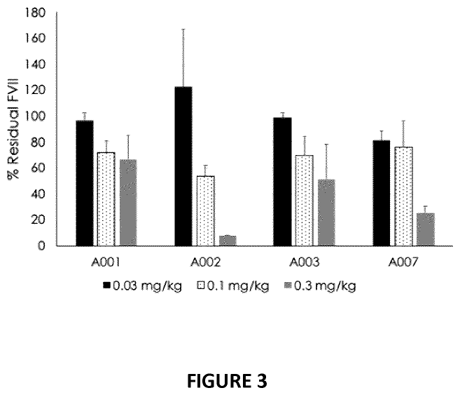

The activity of the ionizable lipids in vitro was evaluated in luciferase expressing human prostate cells (22Rv1). LNPs containing siRNA against firefly luciferase were prepared as described in Example 2. Cells were treated with 0.1-1 μg/mL of LNPs containing siRNA for 16-24 hr and then lysed with Glo-Lysis Buffer (Promega). Equal amounts of Steady-Glo reagent (Promega) was added to each sample and the level of luminescence was determined using a Synergy LX plate reader (BioTek). FIG. 2A shows the luminescence levels of various treatments. In general, a dose-dependent effect in silencing of the firefly luciferase gene was observed. FIG. 2B shows the relative activity of formulations containing ionizable lipids A001-A007. The results show that the ionizable lipids of the disclosure can effectively deliver siRNA and induce gene-silencing in vitro. A007 was the most active at 1 ug/mL in this in vitro model of gene silencing.

### Example 4: Determination of LNP Activity in the Mouse Factor VII Model

The activity of the ionizable lipids in vivo was next evaluated in a mouse FVII model. The results show that the ionizable lipids of the disclosure can effectively deliver nucleic acids in vivo.

LNPs prepared as described in Example 2 containing siRNA against factor VII (FVII) were diluted with PBS such that injection volumes were maintained at 10 mL/kg body weight and administered (based on siRNA concentration) intravenously via tail vein in 6 to 8 weeks old female C57Bl/6 mice (Charles River Laboratories, Wilmington, Mass.). At 24 hours post-injection, animals were euthanized and blood was collected via intracardiac sampling. Blood samples were allowed to coagulate at 4° C. overnight and the serum was separated followed by centrifugation for 15 min at 12,000 rpm. The serum FVII levels were determined using the Biophen VII chromogenic assay (Aniara, Mason, Ohio) according to the protocol of the manufacturer.

FIG. 3 shows the residual FVII levels in mice injected with INT-A001, INT-A002, INT-A003, INT-A005, or INT-A007 formulations. It was determined that these ionizable lipids were active in mediating gene-silencing. INT-A002 was the most active of the formulated lipids tested. The ED 50 for INT-002 was estimated as less than 0.1 mg/kg (ED 50 is the effective dose to achieve 50% gene-silencing).

### Example 5: Formulation of Lipid Nanoparticles Containing mRNA into LNP

The ability of formulations containing INT-A001, INT-A002, INT-A003 and INT-A004 to deliver mRNA in vitro and in vivo was assessed. The results show that these ionizable lipids can effectively deliver mRNA.

LNPs were prepared as described in Example 2 containing Firefly Luciferase mRNA. As shown in Table 3 below, the INT-A001, INT-A002, INT-A003, and INT-A004 ionizable lipids facilitated the incorporation of Luciferase mRNA at high encapsulation efficiency and low polydispersity, both of which are desirable physiochemical properties for drug delivery systems.

TABLE 3
Physiochemical parameters of LNP containing ionizable lipids and mRNA

| Ionizable | Particle Diameter | Polydispersity index | Encapsulation |
|---|---|---|---|
| Lipid ID | (nm) | (PDI) | Efficiency (%) |
| INT-A001 | 43 | 0.064 | 96 |
| INT-A002 | 47 | 0.053 | 99 |
| INT-A003 | 49 | 0.050 | 99 |
| INT-A004 | 42 | 0.090 | 96 |

The activity of the ionizable lipids in vitro was evaluated in cultured HepG2 cells. LNPs containing Firefly Luciferase mRNA were prepared as described in Example 2. LNPs were diluted to 0.125-1 μg/mL with DMEM media containing 10% FBS and incubated with HepG2 cells for 16-24 hr. Cells were then lysed with Glo-Lysis Buffer (Promega). Equal amounts of Steady-Glo reagent (Promega) was added to each sample and the level of luminescence was determined using a Synergy LX plate reader (BioTek). FIG. 4A shows the luminescence levels of various treatments. INT-A003 formulation was the most active in delivering mRNA and inducing its expression in vitro.

To assess in vivo activity, LNPs containing Firefly Luciferase mRNA were prepared as described in Example 2. LNPs were diluted with PBS and administered at 1 mg/kg intravenously via tail vein in 6 to 8 weeks old female C57Bl/6 mice (Charles River Laboratories, Wilmington, Mass.). At 4 hours post-injection, animals were euthanized and the livers were collected. Approximately 100 mg of liver was homogenized in 0.5 mL Glo Lysis Buffer (Promega). The homogenate was further diluted 1:4 with the lysis buffer and then 50 μL of diluted homogenate was added to 50 μL Steady-Glo reagent (Promega). The level of luminescence was determined using a Synergy LX plate reader (BioTek). FIG. 4B shows the relative level of luminescence as a result of successful delivery of mRNA in vivo. INT-A002 formulation was the most active in delivering mRNA and inducing mRNA expression in vivo.

### Example 6: Formulation of Lipid Nanoparticles Containing Antisense Oligonucleotides into LNPs

LNPs were prepared as described in Example 2 containing antisense oligonucleotide. As shown in Table 4 below, the INT-A001, INT-A002, INT-A003, INT-A004, INT-A005, INT-A006 and INT-A007 ionizable lipids facilitated the incorporation of antisense oligonucleotide at high encapsulation efficiency and low polydispersity, both of which are desirable physiochemical properties for drug delivery systems.

TABLE 4
Physiochemical parameters of LNP containing ionizable lipids and
antisense oligonucleotide

| Ionizable | Particle Diameter | Polydispersity | Encapsulation |
|---|---|---|---|
| Lipid ID | (nm) | index (PDI) | Efficiency (%) |
| INT-A001 | 44 | 0.103 | 84 |
| INT-A002 | 49 | 0.096 | 92 |
| INT-A003 | 54 | 0.036 | 89 |
| INT-A004 | 45 | 0.065 | 88 |
| INT-A005 | 47 | 0.090 | 74 |
| INT-A006 | 55 | 0.062 | 88 |
| INT-A007 | 55 | 0.070 | 84 |

### Example 7: Formulation of Lipid Nanoparticles Containing Hyaluronic Acid (HA) into LNPs

Hyaluronic acid was used as an example of anionic cargos. LNPs were prepared as described in Example 2 containing HA with a molecular weight of 8-15 kDa. As shown in Table 5 below, the INT-A002, INT-A003, INT-A005, INT-A006, and INT-A007 ionizable lipids facilitated the incorporation of HA.

TABLE 5
Physiochemical parameters of LNP containing ionizable lipids and HA

| Ionizable | Particle Diameter | Polydispersity index | HA:Lipid |
|---|---|---|---|
| Lipid ID | (nm) | (PDI) | (wt/wt) |
| INT-A002 | 83 | 0.027 | 0.093 |
| INT-A003 | 64 | 0.035 | 0.094 |
| INT-A005 | 71 | 0.096 | 0.114 |
| INT-A006 | 74 | 0.037 | 0.078 |
| INT-A007 | 58 | 0.065 | 0.096 |

### Example 8: Formulation of Lipid Nanoparticles Containing Acidic Peptides into LNPs

Similar to nucleic acids, proteins or peptides with a net negative charge can be incorporated into LNP using cationic ionizable lipids. LNPs were prepared as described in Example 2 containing an acidic peptide with a molecular weight of 4.2 kDa and a predicted net charge of −3. As shown in Table 6 below, the INT-A001, INT-A002, INT-A003, INT-A004, INT-A005, INT-A006, and INT-A007 ionizable lipids facilitated the incorporation of this acidic peptide.

TABLE 6
Physiochemical parameters of LNP containing ionizable lipids and a
4.2 kDa acidic peptide

| Ionizable | Particle Diameter | Polydispersity index | Peptide:Lipid |
|---|---|---|---|
| Lipid ID | (nm) | (PDI) | (wt/wt) |
| INT-A001 | 48 | 0.021 | 0.161 |
| INT-A002 | 62 | 0.015 | 0.157 |
| INT-A003 | 76 | 0.031 | 0.160 |
| INT-A004 | 40 | 0.034 | 0.158 |
| INT-A005 | 87 | 0.021 | 0.193 |
| INT-A006 | 58 | 0.070 | 0.132 |
| INT-A007 | 52 | 0.049 | 0.162 |

### Example 9: Formulation of Lipid Nanoparticles Containing Small Acidic Peptides into LNPs

LNPs were prepared as described in Example 2 containing an acidic peptide with a molecular weight of 2.0 kDa and a predicted net charge of −3. As shown in Table 7 below, INT-A003, INT-A004, INT-A006, and INT-A007 ionizable lipids facilitated the incorporation of this acidic peptide.

TABLE 7
Physiochemical parameters of LNP containing ionizable lipids and a
2.0 kDa acidic peptide

| Ionizable | Particle Diameter | Polydispersity index | Peptide:Lipid |
|---|---|---|---|
| Lipid ID | (nm) | (PDI) | (wt/wt) |
| INT-A003 | 142 | 0.025 | 0.074 |
| INT-A004 | 59 | 0.051 | 0.073 |
| INT-A006 | 94 | 0.054 | 0.061 |
| INT-A007 | 79 | 0.019 | 0.075 |

### Example 10: Formulation of Lipid Nanoparticles Containing Cationic Cargo into LNPs

A basic peptide was used as an example of cationic cargos. LNPs were prepared as described in Example 2 containing a basic peptide with a molecular weight of 2.0 kDa and a predicted net charge of +4. As shown in Table 8 below, INT-A008 and INT-A009 ionizable lipids facilitated the incorporation of this basic peptide.

TABLE 8
Physiochemical parameters of LNP containing ionizable lipids and a
2.0 kDa basic peptide

| Ionizable | Particle Diameter | Polydispersity index | Peptide:Lipid |
|---|---|---|---|
| Lipid ID | (nm) | (PDI) | (wt/wt) |
| INT-A008 | 39 | 0.058 | 0.104 |
| INT-A009 | 39 | 0.057 | 0.121 |

The claims appended hereto should not be limited by any of the specific embodiments set forth above and shall be construed to include all possible embodiments and equivalents to which such claims are entitled.

1. A lipid comprising a branched lipid moiety L having the structure of Formula I:
A-(V)ₘ—Z-L, wherein Formula I:
A is a head group that is ionizable, permanently charged or zwitterionic;
(V)ₘ is an optional —(CR1R2)ₘ-, and m is 1 to 10 or 2 to 6, wherein R1 and R2 are each independently: hydrogen, optionally substituted alkyl, alkenyl, alkynyl, aryl, cycloalkyl, cycloalkylalkyl, or a heterocycle; and
Z-L has a structure of Formula II, IIa or IIb below,
and wherein L is a hydrocarbon structure and has a moiety of Formula IIIc below: 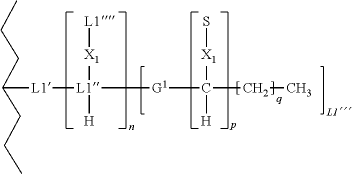
wherein a scaffold carbon chain of L is denoted by L1′-L1″-CH—[CH₂]_{q}—CH₃, and wherein
the total number of carbon atoms in the L carbon backbone is 10 to 30;
L1′ is a linear hydrocarbon chain having no heteroatoms and has 5-12 carbon atoms and 0-3 cis or trans double bonds;
L1″ is a carbon atom;
each X1 is independently selected from an ether, ester and carbamate group;
L1′″ is a carbon backbone portion of the scaffold carbon chain L and is depicted by G¹-CH—[CH₂]_{q}—CH₃ and wherein G′ is a hydrocarbon chain of 0-4 carbon atoms, optionally having one cis or trans double bond;
wherein n is 0 to 4;
wherein p is 1 to 4;
wherein n+p is 1 to 4;
q is 0 to 20;
wherein each S and L1″″ is a hydrocarbon side chain and is independently:
(a) a linear or branched terminating hydrocarbon chain with 0 to 5 cis or trans C═C and 2 to 30 carbon atoms and conjugated to one of a respective X1 at any carbon atom in its hydrocarbon chain thereof; or
(b) a branched hydrocarbon structure of Formula IIIc,
wherein each one of the L1″″ and S hydrocarbon chains in the lipid moiety is optionally substituted with a heteroatom, with the proviso that no more than 2 heteroatoms are substituted in the hydrocarbon chains,
Formula II linear linker structure:
X1-L_{b},
wherein X1 is optional and X1 is selected from an ether, ester and carbamate group; and
L_{b} is a branched lipid of Formula IIIc;
Formula IIa branched linker structure: 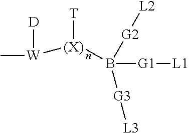
W is optional;
W, if present, is an X1 linkage, N—C(O), N—C(O)O, or N—OC(O);
wherein W is optionally substituted with D, which is an optionally substituted alkyl, alkenyl, alkynyl, aryl, cycloalkyl, cycloalkylalkyl, or heterocycle;
each occurrence of (X)ₙ is an independently selected —(CR1R2)ₙ-; n of (X)ₙ is 0 to 10; and T is optional and is an alkyl, alkenyl, alkynyl, aryl, cycloalkyl, cycloalkylalkyl, or heterocycle and wherein T is optionally substituted;
B is a carbon atom linked to L1 and L2 via respective G1 and G2;
wherein G1 and G2 are independently selected from an X1;
wherein each of G1 and G2 is independently optionally covalently bonded to B via an intervening (G)ᵤ group as B-(G)ᵤ-G1 or B-(G)ᵤ-G2, respectively;
wherein (G)ᵤ is an independently selected —(CR1R2)ᵤ- wherein R1 and R2 are each independently:
hydrogen, optionally substituted alkyl, alkenyl, alkynyl, aryl, cycloalkyl, cycloalkylalkyl, or heterocycle and u is 0 to 16;
wherein G3 is optional and is selected from X1 and optionally covalently bonded to the B via an intervening (G)ᵤ group as B-(G)ᵤ-G3;
L1 is a branched hydrocarbon of Formula IIIc;
L2 is a hydrocarbon chain having 1 to 20 carbon atoms and 0 to 2 cis or trans double bonds or has the structure of Formula IIIc;
L3 if present is hydrogen, a linear or branched hydrocarbon chain having 1 to 20 carbon atoms and 0 to 2 cis or trans double bonds or has the structure of Formula IIIc;
Formula IIb ring structure: 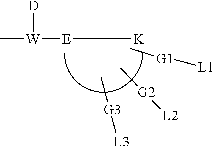
wherein the curved line represents a ring and E and K depict atoms that partially form the structure of the ring, which ring is a substituted or unsubstituted ring having 3 to 8 ring atoms;
wherein at least one of L1, L2 and L3 are bonded to a single atom in the ring, optionally via a respective G1, G2 and G3, wherein each of G1, G2 and G3 is independently optionally covalently bonded to a respective one of the L1, L2 and L3 via an intervening (G)ᵤ, as G1-(G)ᵤ-L1, G2-(G)ᵤ-L2 or G3-(G)ᵤ-L3, respectively;
wherein L1 and optionally L2 and/or L3 of Formula IIb have the structure of Formula IIIc; and
wherein the A moiety is selected from one of:
(i) ionizable cationic moieties selected from the group consisting of: 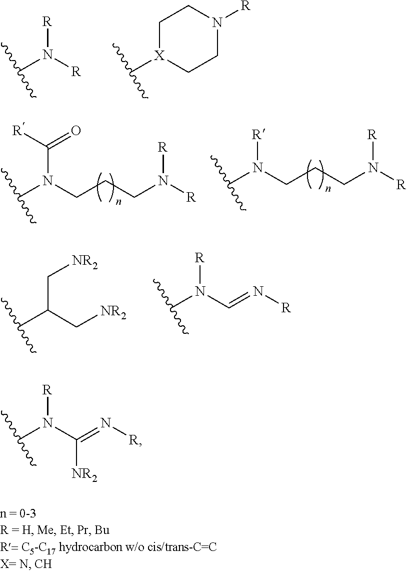
(ii) permanently charged moieties selected from the group consisting of: 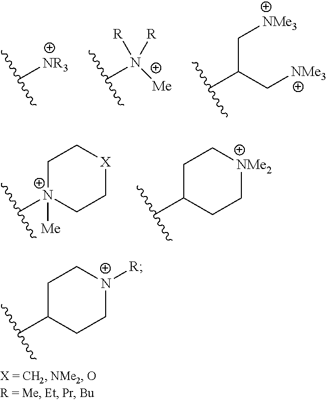
(iii) ionizable anionic moieties selected from the group consisting of: 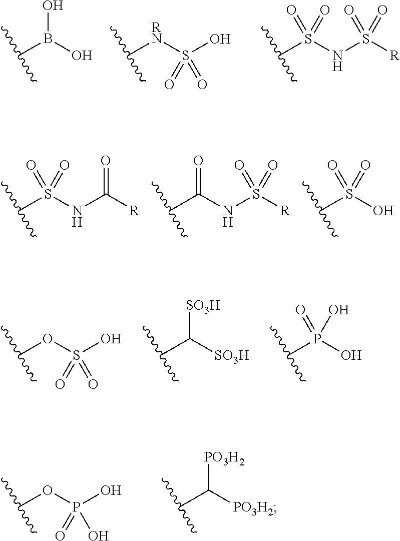
or
(iv) zwitterionic moieties selected from the group consisting of:

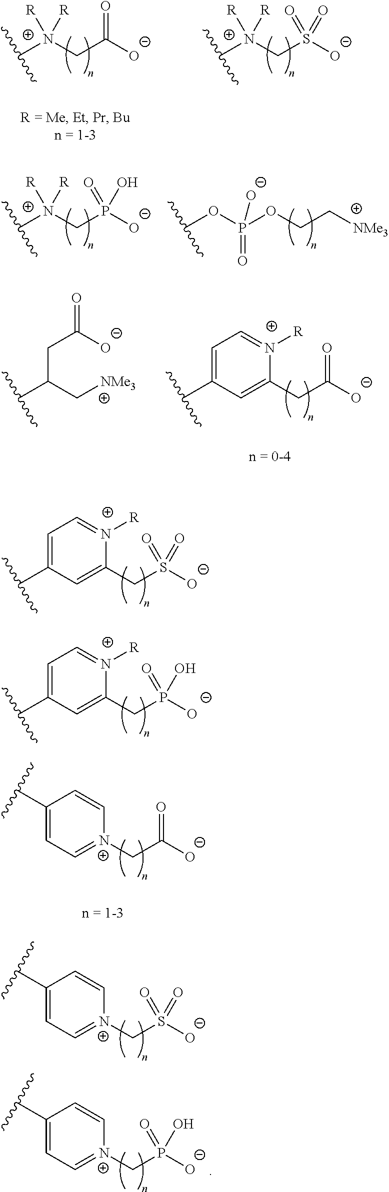

2. The lipid of claim 1, wherein Z-L has the structure of Formula II (linear linker structure):
X1-L_{b};
wherein L1′ of Formula IIIc has 5 to 9 carbon atoms and has 0 to 2 cis or trans double bonds;
wherein G¹ of Formula IIIc is absent, CH₂ or CH₂CH═CH, and wherein the double bond is cis or trans;
wherein L1″″, if present, and S of Formula IIIc are independently selected from a hydrocarbon with 0-5 cis or trans CH═CH and 2 to 18 carbon atoms;
and
wherein q is 1 to 9.

3. The lipid of claim 1, wherein (V)ₘ is (CH₂)ₘ, wherein m is 1 to 20;
Z-L has the structure of Formula IIa (branched linker structure): 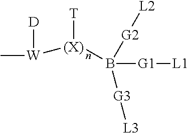
wherein W is an ether, ester or carbamate group and D is absent, and (X)ₙ is (CH₂)ₙ, wherein n is 1 to 10;
wherein G1 and G2 are present and are covalently bonded to the B via a (G)ᵤ, as B-(G)ᵤ-L1 or B-(G)ᵤ-L2, wherein (G)ᵤ is (CH₂)ᵤ;
wherein G3-L3 is present and is a hydrocarbon selected from CH₃ and CH₂CH₃; or wherein G3-L3 is CH₂X1L3 and L3 is a linear or branched hydrocarbon chain having 1 to 20 carbon atoms and 0 to 2 cis or trans double bonds or has the structure of Formula IIIc.

4. The lipid of claim 1, wherein Z-L has the structure of Formula IIb:
Formula IIb ring structure: 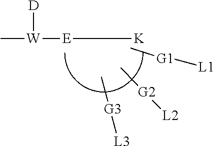
wherein the curved line represents a ring and E and K depict atoms that partially form the structure of the ring, which ring is a substituted or unsubstituted carbon ring having 3 to 6 ring atoms.

5. The lipid of claim 4, wherein the ring comprises 3 or 5 carbon atoms.

6. The lipid of claim 4, wherein at least L1 and L2 are present and are attached to the ring via respective G1 and G2 groups and wherein each G1 and G2 group is optionally covalently bonded to an atom of the ring via an intervening (G)ᵤ, wherein (G)ᵤ is (CH₂)ᵤ and u is 0 to 10 or 0 to 6.

7. The lipid of claim 1, wherein the lipid is capable of assembling into a lipid nanoparticle in combination with other lipids in aqueous solution.

8. The lipid of claim 7, wherein the other lipids are vesicle forming lipids and include phosphatidylcholine, phosphatidylglycerol, phosphatidylserine, phosphatidylethanolamine, phosphatidic acid, ceramides, sphingomyelin or a hydrophilic polymer-lipid conjugate.

9. The lipid of claim 1, wherein the R1 or R2 of (V)ₘ is the cycloalkyl that is an optionally substituted mono-, bi-, or tri-cyclic carbon ring.

10. The lipid of claim 1, wherein the R1 or R2 of (V)ₘ are each independently: selected from the heteroatom ring having 4 to 12 ring atoms.

11. A drug delivery vehicle formulation comprising the lipid of claim 1 incorporated in a lipid bilayer or monolayer thereof and comprising cargo molecule or compound that is a nucleic acid, protein or a peptide.

12. The drug delivery formulation of claim 11, wherein the nucleic acid is a small interfering RNA, a small activating RNA, a messenger RNA, a microRNA, an antisense oligonucleotide, a ribozyme, an aptamer, a plasmid, a circular DNA, a linear DNA, an antagomir, an anti-miRNA oligonucleotide or an miRNA mimic.

13. The drug delivery formulation of claim 11, wherein the cargo molecule or compound is a peptide.

14. The drug delivery vehicle formulation of claim 11 comprising a lipid nanoparticle (LNP).

15. The drug delivery vehicle formulation of claim 14, wherein the polydispersity of the lipid nanoparticle is less than 0.15.

16. The drug delivery vehicle formulation of claim 14, wherein the encapsulation efficiency of the lipid nanoparticle is 70% to 100%.

17. The drug delivery vehicle formulation of claim 16, wherein the encapsulation efficiency of the lipid nanoparticle is 80% to 100%.
